SEQUENCE LISTING (3 sequences)

SEQ ID NO: 1 - DNA, 10011 bp, Homo sapiens
ttcctccgcgaaggctcctttgatattaatagtgttggtgtcttgaaactgacgtaatgcgcggagactgaggtcctgacaagcgataacatttctgataaagacccgatcttactgcaatctctagcgtcctcttttttggtgctgctggtttctccagacctcgcgtcctctcgattgctctctcgccttcctatttcttttttttttttttaaacaaaaaacaacaccccctcccctctcccacccggcaccgggcacatccttgctctatttcctttctctttctctctctctctctctcttttttaataagggtgggggagggaaaggggggggaggcaggaaagacctttttctctcccccccgcaataatccaagatcaactctgcaaacaacagaagacggttcatggctttggccgccgcgccaccatctttcgggctgccgagggtgttcttgacgattaatcaacagatgtacagatcagctctcaaaatgtcttctgtgtcttctgagcgtcttctaagacaattgcattagcctcctgctagttgactaatagaattaataattgtaaaaagcactctaaagccacatgccttatgaagtcaatgctgggtatgattttacaaatatggtccggaaaaagaacccccctctgagaaacgttgcaagtgaaggcgagggccagatcctggagcctataggtacagaaagcaaggtatctggaaagaacaaagaattctctgcagatcagatgtcagaaaatacggatcagagtgatgctgcagaactaaatcataaggaggaacatagcttgcatgttcaagatccatcttctagcagtaagaaggacttgaaaagcgcagttctgagtgagaaggctggcttcaattatgaaagccccagtaagggaggaaactttccctcctttccgcatgatgaggtgacagacagaaatatgttggctttctcatttccagctgctgggggagtctgtgagcccttgaagtctccgcaaagagcagaggcagatgaccctcaagatatggcctgcaccccctcaggggactcactggagacaaaggaagatcagaagatgtcaccaaaggctacagaggaaacagggcaagcacagagtggtcaagccaattgtcaaggtttgagcccagtttcagtggcctcaaaaaacccacaagtgccttcagatgggggtgtaagactgaataaatccaaaactgacttactggtgaatgacaacccagacccggcacctctgtctccagagcttcaggactttaaatgcaatatctgtggatatggttactacggcaacgaccccacagatctgattaagcacttccgaaagtatcacttaggactgcataaccgcaccaggcaagatgctgagctggacagcaaaatcttggcccttcataacatggtgcagttcagccattccaaagacttccagaaggtcaaccgttctgtgttttctggtgtgctgcaggacatcaattcttcaaggcctgttttactaaatgggacctatgatgtgcaggtgacttcaggtggaacattcattggcattggacggaaaacaccagattgccaagggaacaccaagtatttccgctgtaaattctgcaatttcacttatatgggcaactcatccaccgaattagaacaacattttcttcagactcacccaaacaaaataaaagcttctctcccctcctctgaggttgcaaaaccttcagagaaaaactctaacaagtccatccctgcacttcaatccagtgattctggagacttgggaaaatggcaggacaagataacagtcaaagcaggagatgacactcctgttgggtactcagtgcccataaagcccctcgattcctctagacaaaatggtacagaggccaccagttactactggtgtaaattttgtagtttcagctgtgagtcatctagctcacttaaactgctagaacattatggcaagcagcacggagcagtgcagtcaggcggccttaatccagagttaaatgataagctttccaggggctctgtcattaatcagaatgatctagccaaaagttcagaaggagagacaatgaccaagacagacaagagctcgagtggggctaaaaagaaggacttctccagcaagggagccgaggataatatggtaacgagctataattgtcagttctgtgacttccgatattccaaaagccatggccctgatgtaattgtagtggggccacttctccgtcattatcaacagctccataacattcacaagtgtaccattaaacactgtccattctgtcccagaggactttgcagcccagaaaagcaccttggagaaattacttatccgtttgcttgtagaaaaagtaattgttcccactgtgcactcttgcttctgcacttgtctcctggggcggctggaagctcgcgagtcaaacatcagtgccatcagtgttcattcaccacccctgacgtagatgtactcctctttcactatgaaagtgtgcatgagtcccaagcatcggatgtcaaacaagaagcaaatcacctgcaaggatcggatgggcagcagtctgtcaaggaaagcaaagaacactcatgtaccaaatgtgattttattacccaagtggaagaagagatttcccgacactacaggagagcacacagctgctacaaatgccgtcagtgcagttttacagctgccgatactcagtcactactggagcacttcaacactgttcactgccaggaacaggacatcactacagccaacggcgaagaggacggtcatgccatatccaccatcaaagaggagcccaaaattgacttcagggtctacaatctgctaactccagactctaaaatgggagagccagtttctgagagtgtggtgaagagagagaagctggaagagaaggacgggctcaaagagaaagtttggaccgagagttccagtgatgaccttcgcaatgtgacttggagaggggcagacatcctgcgggggagtccgtcatacacccaagcaagcctggggctgctgacgcctgtgtctggcacccaagagcagacaaagactctaagggatagtcccaatgtggaggccgcccatctggcgcgacctatttatggcttggctgtggaaaccaagggattcctgcagggggcgccagctggcggagagaagtctggggccctcccccagcagtatcctgcatcgggagaaaacaagtccaaggatgaatcccagtccctgttacggaggcgtagaggctccggtgttttttgtgccaattgcctgaccacaaagacctctctctggcgaaagaatgcaaatggcggatatgtatgcaacgcgtgtggcctctaccagaagcttcactcgactcccaggcctttaaacatcattaaacaaaacaacggtgagcagattattaggaggagaacaagaaagcgccttaacccagaggcacttcaggctgagcagctcaacaaacagcagaggggcagcaatgaggagcaagtcaatggaagcccgttagagaggaggtcagaagatcatctaactgaaagtcaccagagagaaattccactccccagcctaagtaaatacgaagcccagggttcattgactaaaagccattctgctcagcagccagtcctggtcagccaaactctggatattcacaaaaggatgcaacctttgcacattcagataaaaagtcctcaggaaagtactggagatccaggaaatagttcatccgtatctgaagggaaaggaagttctgagagaggcagtcctatagaaaagtacatgagacctgcgaaacacccaaattattcaccaccaggcagccctattgaaaagtaccagtacccactttttggacttccctttgtacataatgacttccagagtgaagctgattggctgcggttctggagtaaatataagctctccgttcctgggaatccgcactacttgagtcacgtgcctggcctaccaaatccttgccaaaactatgtgccttatcccaccttcaatctgcctcctcatttttcagctgttggatcagacaatgacattcctctagatttggcgatcaagcattccagacctgggccaactgcaaacggtgcctccaaggagaaaacgaaggcaccaccaaatgtaaaaaatgaaggtcccttgaatgtagtaaaaacagagaaagttgatagaagtactcaagatgaactttcaacaaaatgtgtgcactgtggcattgtctttctggatgaagtgatgtatgctttgcatatgagttgccatggtgacagtggacctttccagtgcagcatatgccagcatctttgcacggacaaatatgacttcacaacacatatccagaggggcctgcataggaacaatgcacaagtggaaaaaaatggaaaacctaaagagtaaaaccttagcacttagcacaattaaatagaaataggttttcttgatgggaattcaatagcttgtaatgtcttatgaagacctattaaaaaaatacttcatagagcctgccttatccaacatgaaattcccttcttttgttattctttcttttgatgagtaggttaccaagattaaaaagtgagataaatggtcaatgagaaagaatggaagatggtaaacaatcactttttaaaacctgttaagtcaaaaccatcttggctaatatgtactggggaaataatccataagagatatcaccagactagaattaatatatttataaagaaagagaccaaaactgtctagaatttgaaagggtttacatattattatactaaagcagtactggactggccattggaccatttgttccaaaacccataaattgttgcctaaatttataatgatcatgaaaccctaggcagaggaggagaaattgaaggtccagggcaatgaaagaaaaatggcgccctctcaatttagtcttctctcattggccatgtttcagattttgacctagaaatgcgagctgtggttaggcttggttagagtgcagcaagcaacatgacagatggtggcacgctgtttttacccagccctgcctgtacatacacatgcacaccctctctgatatttttgtcctttagatgttcaaatactcagtagtccttttgtttgcggtttagattcattttgtccacacatgtacccattttaaaaaacaatgtcctcgatgcttctgtagtgatttcattttagccaggtatttctttcttgtgtgtgatgaaccagtatggatttgcttttctaagcctcctgttggttactaatctcacttggcacattataactaaaggaatcccctcaattcaaaagcatagatggatacaaatgtcagaccgtgggtttaatttgtttagaacacatggcatttcttcacaaggtaacctgctgtatttatttattttcttttggttaaatataatttccaaactttgtggtcaggcagcgtctaaggttacgttaccacagactgacagttggtatatgtaccagccaatcccttcattaaatgtatacagatttagttaagtagcattaaataggattcttagaagtatgtcctcatagaacttttaatacttaaggctttgtaaaaactatccatgaagggaaagctcctcagcataactgctcagggaaatagggctaaataactgaacattaaataattggttaaaggtgctgttagtcgagcctcaatgcttgctacaaggatgtatgtacaaggactgactttaataatttgcattatattgtcccaaccagtagtttattttttgccacggagatgtagaagatattacaagctactggatgcactgtcagattaacttatttcattaaagaagttgggagaacaaataggaaaaaaaaaacttatttttctagtaaatattaatgtattacatttcaaataatggtgcctgacatattgaataattattttctacagtgtacgtatgcaacaaagatattccatcatgcattagagtcagttctggctctgcctagctgtttacatttgcaaatgtagcaaacaaggtaatgaagcaactatttctattgcagtagatatccttttgtgtgtgtgtgtgtgcattaaagttgtaaacggtaacatgaaacaaatgaaagttcttgctataatggtatggaaaacaagaaggaaatgaaaatatttttatgcctacttaggaaaaaaagggtagcacttattcattccaagtacttttttttttttaatttttaagctcttaactcacattgttatgcttaagatgataaacatatatcctctttttattgctttgtctatgtttcatatgaaacatttcagaaattattttgataagtgttgctggaatctgcaacgctgatttttttttgcattctgtagtcgcatttgcactccatttttacattaattcgcagttgctttgtatcattgttttgtttgggttttgtttctttttcacagtgccgggtcttcgtttcttaaagttggatggcaggtagagttcaaccagttcgtgactgttgtagcgaatgaagttaaaaaaatgtctttctgatgttgtgttgtcattttcatttttgcatttttttgtttgcatattaaaaaaagagaaaagagaaagcaagagacagaaatcaggactaagtcctctgcttcagtttcattgttaacgggccttattctgatctcacctgtcgcgtagctctaatattcacataaactgaaataaagaagtggaatgaggagctttgacattcaaattatgtgatgtaatttatcttccttaggaattttgatggatgcatctcaaaatgtatagccagacttgagaggtgacaattaaagatctaaaaaagagaggagattcccccaaacaacaatatttaattttcttagtaaaaagaataacagaatgcatcgtggcaatccttaagcaacattatctatgtggactgcttaaatcagcaaaacaccagaagtttggttaacttgggcaatatgacaagtattactttttgggcaaaactactcattaagcaatttctctagtgtgtcggacacaaataggttctttatttttggcatgtatgcctttttattttcattcaattttttttttttctcagacagacatagtagtatcaactagcattggaaaatacatatcactattcttggaatatttatggtcagtctactttttagtaaaatatttttggatagcgttgacacgatagatcttattccatacttctttattattgataattttattttcattttttgctttcattattatacatattttggtggagaagaggttgggcttttttgaaagagacaaaaatttattataacactaaacactccttttttgacatattaaagcctttattccatctctcaagatatattataaaatttatttttttaatttaagatttctgaattattttatcttaaattgtgattttaaacgagctattatggtacggaactttttttaatgaggaatttcatgatgatttaggaattttctctcttggaaaaggcttcccctgtgatgaaaatgatgtgccagctaaaattgtgtgccatttaaaaactgaaaatattttaaaattatttgtctatattctaaattgagctttggatcaaactttaggccaggaccagctcatgcgttctcattcttccttttctcactctttctctcatcactcacctctgtattcattctgttgtttgggatagaaaaatcataaagagccaacccatctcagaacgttgtggattgagagagacactacatgactccaagtatatgagaaaaggacagagctctaattgataactctgtagttcaaaaggaaaagagtatgcccaattctctctacatgacatattgagattttttttaatcaacttttaagatagtgatgttctgttctaaactgttctgttttagtgaaggtagatttttataaaacaagcatggggattcttttctaaggtaatattaatgagaagggaaaaaagtatctttaacagctctttgttgaagcctgtggtagcacattatgtttataattgcacatgtgcacataatctattatgatccaatgcaaatacagctccaaaaatattaaatgtatatatattttaaaatgcctgaggaaatacatttttcttaataaactgaagagtctcagtatggctattaaaataattattagcctcctgttgtgtggctgcaaaacatcacaaagtgaccggtcttgagacctgtgaactgctgccctgtttagtaaataaaattaatgcatttctagagggggaatatctgccatccagtggtggaaatgtggagtaaagaagctggtggtctgcttctgtgctgtatgccagccttttgccttaagttgagaggaggtcaactttagctactgtctttggtttgagagccatggcaaaaaaaaaaaaagaaaaaaagatcaagtcgtctttggtgagccagtaaggtgaaagcttgctgactgtccaaggcacaagagaaaattgaggaattgaaatgcaacctgagtatcaaactaaatattctaatcaaaggtaggtactgttaggtggaattctatcagcaggcaactgcaaatgagaagaagatagaaggacgcccgtcgggactttggagggcattgttattttcccaaagaaagacggccaagggcagaggcatggattctttgcagagcacttccttttggtttttcagtactgtttcatagacagtgggctcacatgttcctgatagtgctgcagttgcttagaaagcatcccagttaattgcagtaattagaacttctggaatatgctagggcagaagtatgtcaagtatgtcacatgaagaaaatgtgaaattcaagagtaatccacacgtgagaaactagacaatgtacattcatgtgttctcttgaaaggaaagggagagctgtaagcttcactctgtcctacaccggagaaaagcaggaataactttaccgtggaaataatgtttagcttttatcagagaaaattgtccttctagagcatagagtcccaaaactcaattctggttttcccctgttttttttttttttttttttcccaacatatgaactgcagcatatcactttttctttttgtgcctcaggttcctcacctgtaaaattgaaaaatatatgtattaataatattattaataataataatggtaatgtagtacttgtttgtaaagcactttgagatccttggttgaaaggcaccataggagtgccaagtattattatgtggccaagggggttatttaaactgtcagttcccaaaggccaggaaaggttggggtcatttttcttaaagacgagctgtaaatatcaactaggcagccaatagtgttgactatgaagatgcaaaactattactaggctgataaaatcatagtttcttaatggctaccaataaggcaaatatcacaataataaacgccaaattccttagggcggactatttgacaaccacatggaaaactttgggggaggcatgaggggggaacatctcaaaatgccaatgtaaaatttaacttacagcaatattcaccagcagaaaatgtctttcatatggaatgatttcatgttgctaagaaaaagaattcaatttgtagtcctgatttgaatactagaatgttggctataatagttctgttcttacaacacatgaaattttttcgttttattttattttgttttcatagtgcatgttcatttctactcacaaacatgttcttggtgtatttcttatgcaaacaatcttcaggcagcaaagatgtctgttacatctaaacttgaataataaagttttaccaccagttacaca

SEQ ID NO: 2 - protein, 1281 aa, Homo sapiens
MVRKKNPPLRNVASEGEGQILEPIGTESKVSGKNKEFSADQMSENTDQSDAAELNHKEEHSLHVQDPSSSSKKDLKSAVLSEKAGFNYESPSKGGNFPSFPHDEVTDRNMLAFSSPAAGGVCEPLKSPQRAEADDPQDMACTPSGDSLETKEDQKMSPKATEETGQAQSGQANCQGLSPVSVASKNPQVPSDGGVRLNKSKTDLLVNDNPDPAPLSPELQDFKCNICGYGYYGNDPTDLIKHFRKYHLGLHNRTRQDAELDSKILALHNMVQFSHSKDFQKVNRSVFSGVLQDINSSRPVLLNGTYDVQVTSGGTFIGIGRKTPDCQGNTKYFRCKFCNFTYMGNSSTELEQHFLQTHPNKIKASLPSSEVAKPSEKNSNKSIPALQSSDSGDLGKWQDKITVKAGDDTPVGYSVPIKPLDSSRQNGTEATSYYWCKFCSFSCESSSSLKLLEHYGKQHGAVQSGGLNPELNDKLSRGSVINQNDLAKSSEGETMTKTDKSSSGAKKKDFSSKGAEDNMVTSYNCQFCDFRYSKSHGPDVIVVGPLLRHYQQLHNIHKCTIKHCPFCPRGLCSPEKHLGEITYPFACRKSNCSHCALLLLHLSPGAAGSSRVKHQCHQCSFTTPDVDVLLFHYESVHESQASDVKQEANHLQGSDGQQSVKESKEHSCTKCDFITQVEEEISRHYRRAHSCYKCRQCSFTAADTQSLLEHFNTVHCQEQDITTANGEEDGHAISTIKEEPKIDFRVYNLLTPDSKMGEPVSESVVKREKLEEKDGLKEKVWTESSSDDLRNVTWRGADILRGSPSYTQASLGLLTPVSGTQEQTKTLRDSPNVEAAHLARPIYGLAVETKGFLQGAPAGGEKSGALPQQYPASGENKSKDESQSLLRRRRGSGVFCANCLTTKTSLWRKNANGGYVCNACGLYQKLHSTPRPLNIIKQNNGEQIIRRRTRKRLNPEALQAEQLNKQQRGSNEEQVNGSPLERRSEDHLTESHQREIPLPSLSKYEAQGSLTKSHSAQQPVLVSQTLDIHKRMQPLHIQIKSPQESTGDPGNSSSVSEGKGSSERGSPIEKYMRPAKHPNYSPPGSPIEKYQYPLFGLPFVHNDFQSEADWLRFWSKYKLSVPGNPHYLSHVPGLPNPCQNYVPYPTFNLPPHFSAVGSDNDIPLDLAIKHSRPGPTANGASKEKTKAPPNVKNEGPLNVVKTEKVDRSTQDELSTKCVHCGIVFLDEVMYALHMSCHGDSGPFQCSICQHLCTDKYDFTTHIQRGLHRNNAQVEKNGKPKE

SEQ ID NO: 3 - DNA, 3842 bp, Homo sapiens
tggtccggaaaaagaacccccctctgagaaacgttgcaagtgaaggcgagggccagatcctggagcctataggtacagaaagcaaggtatctggaaagaacaaagaattctctgcagatcagatgtcagaaaatacggatcagagtgatgctgcagaactaaatcataaggaggaacatagcttgcatgttcaagatccatcttctagcagtaagaaggacttgaaaagcgcagttctgagtgagaaggctggcttcaattatgaaagccccagtaagggaggaaactttccctcctttccgcatgatgaggtgacagacagaaatatgttggctttctcatttccagctgctgggggagtctgtgagcccttgaagtctccgcaaagagcagaggcagatgaccctcaagatatggcctgcaccccctcaggggactcactggagacaaaggaagatcagaagatgtcaccaaaggctacagaggaaacagggcaagcacagagtggtcaagccaattgtcaaggtttgagcccagtttcagtggcctcaaaaaacccacaagtgccttcagatgggggtgtaagactgaataaatccaaaactgacttactggtgaatgacaacccagacccggcacctctgtctccagagcttcaggactttaaatgcaatatctgtggatatggttactacggcaacgaccccacagatctgattaagcacttccgaaagtatcacttaggactgcataaccgcaccaggcaagatgctgagctggacagcaaaatcttggcccttcataacatggtgcagttcagccattccaaagacttccagaaggtcaaccgttctgtgttttctggtgtgctgcaggacatcaattcttcaaggcctgttttactaaatgggacctatgatgtgcaggtgacttcaggtggaacattcattggcattggacggaaaacaccagattgccaagggaacaccaagtatttccgctgtaaattctgcaatttcacttatatgggcaactcatccaccgaattagaacaacattttcttcagactcacccaaacaaaataaaagcttctctcccctcctctgaggttgcaaaaccttcagagaaaaactctaacaagtccatccctgcacttcaatccagtgattctggagacttgggaaaatggcaggacaagataacagtcaaagcaggagatgacactcctgttgggtactcagtgcccataaagcccctcgattcctctagacaaaatggtacagaggccaccagttactactggtgtaaattttgtagtttcagctgtgagtcatctagctcacttaaactgctagaacattatggcaagcagcacggagcagtgcagtcaggcggccttaatccagagttaaatgataagctttccaggggctctgtcattaatcagaatgatctagccaaaagttcagaaggagagacaatgaccaagacagacaagagctcgagtggggctaaaaagaaggacttctccagcaagggagccgaggataatatggtaacgagctataattgtcagttctgtgacttccgatattccaaaagccatggccctgatgtaattgtagtggggccacttctccgtcattatcaacagctccataacattcacaagtgtaccattaaacactgtccattctgtcccagaggactttgcagcccagaaaagcaccttggagaaattacttatccgtttgcttgtagaaaaagtaattgttcccactgtgcactcttgcttctgcacttgtctcctggggcggctggaagctcgcgagtcaaacatcagtgccatcagtgttcattcaccacccctgacgtagatgtactcctctttcactatgaaagtgtgcatgagtcccaagcatcggatgtcaaacaagaagcaaatcacctgcaaggatcggatgggcagcagtctgtcaaggaaagcaaagaacactcatgtaccaaatgtgattttattacccaagtggaagaagagatttcccgacactacaggagagcacacagctgctacaaatgccgtcagtgcagttttacagctgccgatactcagtcactactggagcacttcaacactgttcactgccaggaacaggacatcactacagccaacggcgaagaggacggtcatgccatatccaccatcaaagaggagcccaaaattgacttcagggtctacaatctgctaactccagactctaaaatgggagagccagtttctgagagtgtggtgaagagagagaagctggaagagaaggacgggctcaaagagaaagtttggaccgagagttccagtgatgaccttcgcaatgtgacttggagaggggcagacatcctgcgggggagtccgtcatacacccaagcaagcctggggctgctgacgcctgtgtctggcacccaagagcagacaaagactctaagggatagtcccaatgtggaggccgcccatctggcgcgacctatttatggcttggctgtggaaaccaagggattcctgcagggggcgccagctggcggagagaagtctggggccctcccccagcagtatcctgcatcgggagaaaacaagtccaaggatgaatcccagtccctgttacggaggcgtagaggctccggtgttttttgtgccaattgcctgaccacaaagacctctctctggcgaaagaatgcaaatggcggatatgtatgcaacgcgtgtggcctctaccagaagcttcactcgactcccaggcctttaaacatcattaaacaaaacaacggtgagcagattattaggaggagaacaagaaagcgccttaacccagaggcacttcaggctgagcagctcaacaaacagcagaggggcagcaatgaggagcaagtcaatggaagcccgttagagaggaggtcagaagatcatctaactgaaagtcaccagagagaaattccactccccagcctaagtaaatacgaagcccagggttcattgactaaaagccattctgctcagcagccagtcctggtcagccaaactctggatattcacaaaaggatgcaacctttgcacattcagataaaaagtcctcaggaaagtactggagatccaggaaatagttcatccgtatctgaagggaaaggaagttctgagagaggcagtcctatagaaaagtacatgagacctgcgaaacacccaaattattcaccaccaggcagccctattgaaaagtaccagtacccactttttggacttccctttgtacataatgacttccagagtgaagctgattggctgcggttctggagtaaatataagctctccgttcctgggaatccgcactacttgagtcacgtgcctggcctaccaaatccttgccaaaactatgtgccttatcccaccttcaatctgcctcctcatttttcagctgttggatcagacaatgacattcctctagatttggcgatcaagcattccagacctgggccaactgcaaacggtgcctccaaggagaaaacgaaggcaccaccaaatgtaaaaaatgaaggtcccttgaatgtagtaaaaacagagaaagttgatagaagtactcaagatgaactttcaacaaaatgtgtgcactgtggcattgtctttctggatgaagtgatgtatgctttgcatatgagttgccatggtgacagtggacctttccagtgcagcatatgccagcatctttgcacggacaaatatgacttcacaacacatatccagaggggcctgcataggaacaatgcacaagtggaaaaaaatggaaaacctaaagag

(19) United States
(12) Patent Application Publication
(10) Pub. No.: US 2008/0051342 A1
(43) Pub. Date: Feb. 28, 2008

(54) TRPS1-mediated modulation of hair growth

(76) Inventor: Angela M. Christiano, Saddle River, NJ (US)

Correspondence Address: BAKER BOTTS L.L.P., 30 ROCKEFELLER PLAZA, 44TH FLOOR, NEW YORK, NY, 10112-4498, US

(21) Appl. No.: 11/824,193

(22) Filed: Jun. 29, 2007

Related U.S. Application Data
(63) Continuation of application No. PCT/US06/20645, filed on May 26, 2006.
(60) Provisional application No. 60/685,755, filed on May 27, 2005.
(60) Provisional application No. 60/685,754, filed on May 27, 2005.

(51) Int. Cl. A01K 67/00; A61K 31/70; A61K 38/00; A61P 43/00; C12N 5/00; C12N 5/04; C12Q 1/02

(52) U.S. Cl. 514/12; 435/29; 435/325; 435/375; 514/2; 514/23; 514/44; 800/8

(57) ABSTRACT

The present invention provides for methods of inhibiting hair growth, comprising decreasing the level of TRPS1 mRNA and/or protein in hair follicle cells of a subject. The present invention also provides for methods of promoting hair growth, comprising increasing the level of TRPS1 mRNA and/or protein in hair follicle cells of a subject. The level of TRPS1 may be decreased or increased either directly, for example by introducing, into a hair follicle cell, a TRPS1 mRNA or protein. Alternatively, the level of TRPS1 may be decreased or increased indirectly, by providing an agent that results in decreased or increased expression of an endogenous TRPS1 gene. The invention also provides for transgenic animals with aberrancies in TRPS1 expression, and for assay systems (including transgenic animals and cell-based systems) that may be used to identify agents that decrease or increase TRPS1 expression.

### PRIORITY CLAIM

This application claims priority to U.S. Provisional Applications No. 60/685,755 filed May 27, 2005 and No. 60/685,754 filed May 27, 2005, the contents of each of which are incorporated in their entireties herein.

### BACKGROUND OF THE INVENTION

TRPS1 is a nuclear protein of the GATA family, and has nine predicted zinc finger domains, including a single carboxyl-terminal GATA-type zinc finger (Malik et al., 2002, Mol. Cell Biol. 22(24):8592-8600; Malik et al., 2001, EMBO J. 20:1715-1725; Momeni et al., 2000, Nat. Genet. 24:71-74). Members of the GATA family are transcription factors that play important roles in development. Besides the GATA motif, homology between TRPS1 and other proteins is limited to two C-terminal zinc fingers which are closely related to a domain found in the Ikaros family of lymphoid transcription factors (Malik et al., 2002, supra). In contrast to other vertebrate GATA proteins, TRPS1 behaves as a potent, sequence-specific transcriptional repressor in vitro and in vivo, and the repression function maps to the Ikaros domain (Malik et al., 2002, supra; Malik et al., 2001, supra).

Clinically in humans, TRPS1 protein expression is down-regulated by androgens in human prostate cancer (TRPS1 is also known in the art as “GC79”), and analysis of TRPS1 mRNA expression levels in several human tissues showed that the highest levels were observed in normal and tumor breast tissue (Chang et al., 2004, Endocrine-Related Cancer 11:815-822; Chang et al., 2000, J. Natl. Cancer Inst. 92(17):1414-1421). Monoallelic mutations in TRPS1 are associated with the tricho-rhino-phalangeal syndromes (hence, “TRPS”), dominantly inherited conditions characterized by developmental defects in the face, selected bones, and hair, which tends to be sparse and slow growing (Momeni et al., 2000, supra). Deletion of the GATA domain of TRPS1 has been linked to the absence of facial hair (Malik et al., 2002, supra). These clinical observations, as well as the phenotypes of mice carrying TRPS1 mutations, have lead Malik et al. (2002, supra) to suggest that TRPS1 may play a role in hair follicle morphogenesis (see also Kunath et al., 2002, Gene Expr. Patterns 2(1-2):119-122).

TRPS1 resides on human chromosome 8. In addition to tricho-rhino-phalangeal syndrome, several other clinical conditions have been associated with mutations in the region of chromosome 8 occupied by TRPS1. Ambras syndrome, a congenital condition characterized by marked hair overgrowth (hypertrichosis), has been associated with a balanced pericentric inversion in chromosome 8, at or near the location of the TRPS1 gene (Tadin-Strapps et al., 2004, Cytogenet. Genome Res. 107(1-2):68-76; Tadin et al., 2001, Am. J. Med. Genet. 102(1):100-104). In addition, a patient manifesting exostoses, mental retardation and hypertrichosis was found to have a submicroscopic interstitial deletion at chromosome locus 8q24 (Wuyts et al., 2002, Am. J. Med. Genet. 113:326-332).

Prior to the present invention, the fact that certain genetic mutations in the region of TRPS1 result in sparse hair (e.g., tricho-rhino-phalangeal syndrome) while others result in hair overgrowth, or “hypertrichosis” (e.g., Ambras syndrome and the patient described in Wuyts et al., supra), made the role of TRPS1 in hair growth uncertain.

### SUMMARY OF THE INVENTION

The present invention provides for methods and compositions for inhibiting hair growth by decreasing the level and/or activity of TRPS1 mRNA and/or protein. The present invention further provides for methods and compositions for promoting hair growth by increasing the level and/or activity of TRPS1 mRNA and/or protein.

In a first set of embodiments, the present invention provides for methods for identifying agents that may be used to inhibit hair growth, comprising exposing an appropriate test cell or organism to a test agent, and then determining whether expression of TRPS1 is decreased relative to the level of TRPS1 in a control cell or organism not exposed to the test agent. The ability of an agent to decrease TRPS1 indicates that it may be used to inhibit hair growth.

In a second set of embodiments, the present invention provides for methods for identifying agents that may be used to promote hair growth, comprising exposing an appropriate test cell or organism to a test agent, and then determining whether expression of TRPS1 is increased relative to the level of TRPS1 in a control cell or organism not exposed to the test agent. The ability of the agent to increase the level of TRPS1 indicates that the agent may be used to promote hair growth.

In a third set of embodiments, the present invention provides for methods of promoting hair growth comprising increasing the level and/or activity of TRPS1 mRNA and/or protein in cells, preferably hair follicle cells, and more preferably dermal papilla cells, of a subject. The level of TRPS1 may be increased either directly, for example by introducing, into a hair follicle cell, TRPS1 mRNA or protein, or it may be increased indirectly, by providing an agent that results in increased expression of an endogenous TRPS1 gene, increased functional activity of TRPS1 protein, or increased expression of a target of TRPS1.

In a fourth set of embodiments, the present invention provides for methods of inhibiting hair growth in a subject comprising decreasing the level and/or activity of TRPS1 mRNA and/or protein in a cell of the subject. In a particular embodiment, the level and/or activity of TRPS1 is decreased by providing an agent that results in decreased expression of an endogenous TRPS1 gene, decreased functional activity of a TRPS1 protein, or decreased expression and/or activity of a target of a TRPS1 protein. In a particular embodiment, the agent is a catalytic nucleic acid, an antisense oligonucleotide or a siRNA directed to the endogenous TRPS1 gene. In an alternate embodiment, the agent is a catalytic nucleic acid, a siRNA or an antisense oligonucleotide directed to a target of TRPS1, such as Prdm1, Sox 18 or Dkk4, as nonlimiting examples.

In a fifth set of embodiments, the present invention provides for a transgenic non-human animal containing a transgene which interrupts or otherwise disrupts expression of at least one TRPS1 gene, including (i) so-called “knock-out” animals as well as (ii) animals in which the transgene encodes an antisense TRPS1 nucleic acid operably linked to a promoter element, wherein the promoter element may be constitutively active or inducible in hair follicle cells of the animal. Such transgenic animals may be used to study the relationship between TRPS1 expression and hair growth, and may be used in screening methods to identify agents that modulate TRPS1 expression.

In a sixth set of embodiments, the present invention provides for a transgenic non-human animal containing a transgene comprising a TRPS1 gene, operably linked to a promoter element, wherein the promoter element may be constitutively active or inducible in hair follicle cells of the animal. Such transgenic animals may be used to study the relationship between TRPS1 expression and hair growth, and may be used in screening methods to identify agents that increase TRPS1 expression.

In a seventh set of embodiments, the present invention provides for compositions that may be used to inhibit hair growth, which may comprise TRPS1-directed antisense, siRNA, and/or catalytic nucleic acids and/or agents that indirectly inhibit TRPS1 expression.

In an eighth set of embodiments, the present invention provides for compositions that may be used to promote hair growth, which may comprise TRPS1 nucleic acid and/or protein, agents that indirectly modulate TRPS1 expression, and/or hair follicle cells in which TRPS1 expression is increased or which have been administered TRPS1 protein.

In a ninth set of embodiments, the present invention provides for methods of inhibiting hair growth comprising decreasing levels of targets of TRPS1 mRNA and/or protein in cells, preferably hair follicle cells, of a subject. In specific, nonlimiting embodiments, levels of expression of TRPS1 targets, for example, Prdm1, Sox18, and Dkk4, may be decreased by administering, to hair follicle cells, RNAi, antisense oligonucleotide, or catalytic nucleic acids that comprise regions that are complementary to Prdm1, Sox18 or Dkk4 mRNA.

In an tenth set of embodiments, the present invention provides for a method of treating alopecia in a subject comprising administering to cells, preferably hair follicle cells, of the subject, an agent that increases the level of TRPS1 mRNA and/or protein in at least a proportion of cells to which it is administered. In nonlimiting specific embodiments, the alopecia being treated is male pattern baldness in a human.

### BRIEF DESCRIPTION OF THE FIGURES

FIG. 1 shows a schematic representation of the TRPS1 protein. The seven C2H2 type zinc finger domains, a cysteine rich region, a C4 type GATA-like zinc finger domain and a double Ikaros-like zinc finger domain is shown.

FIG. 2A-B show a schematic representation of human chromosome 8, region 8q23 and locus involved in Ambras Syndrome. Known genes and corresponding transcripts (solid horizontal bars with gene symbols) are represented for 8q23 represented from left to right from centromere (CEN) to telomere (TEL) ends. FIG. 2A shows the locus and chromosomal breakpoints in human chromosome 8 corresponding to the Hairy ear (Eh) mutant mouse chromosome 15 inversion. FIG. 2B shows the locus and chromosomal breakpoints in human chromosome 8 corresponding to the Koala (Koa) mutant mouse chromosome 15 inversion. Hairy ears and koala are both mouse mutations with inversions of chromosome 15, corresponding to human chromosome 8. The breakpoints have been deposited in GenBank in accession numbers: AY757365, AY757366, AY757367, and AY757368. Both are characterized by hypertrichosis of hair on the face and ears, similar to human Ambras patients. See Mentzer et al, “Mouse Hairy Ears (Eh) Inversion Mutation Disrupts No Gene Transcripts, But Expression of HOXC Genes In Skin Is Disturbed,” http://www.imgs.org/abstracts/2004abstracts/toc4.shtml.

FIG. 3A-D show images of whole mount in situ hybridization with TRPS1 probe in normal mouse embryos. FIG. 3A, front view, and FIG. 3B, side view, show a mouse embryo stained with antisense TRPS1 probe generated in situ hybridization signals, with staining at phalanges (open arrows), mesenchyme surrounding vibrissae follicles (closed arrows) and snout. FIG. 3C, front view, and FIG. 3D, side view, show a mouse embryo stained with sense TRPS1 probe generated in situ hybridization. No specific signals are seen, demonstrating that staining with antisense probe is specific.

FIG. 4A-D show images of immunofluorescence microscopy visualization of TRPS1 expression in the mouse embryo at various stages during hair follicle morphogenesis. FIG. 4A shows, at e14.0, diffuse, intermittent staining TRPS1 expression in the dorsal epidermis. FIG. 4B shows, at e15.5, TRPS1 expression in nuclei of dermal cells in hair germs, with some diffuse staining still present in epidermis. FIG. 4C, at e16.5 and FIG. 4D, at e17.5, show that TRPS1 expression is restricted to mesenchymal cells surrounding hair follicle and dermal papilla. The dotted line indicates basement membrane.

FIG. 5A-D show immunofluorescence images of TRPS1 expression in the mouse during various stages of postnatal hair follicle cycling. FIG. 5A shows, at P17, that TRPS1 expression is visualized as non-nuclear staining in dermal papilla. FIG. 5B shows, at P22, that TRPS1 expression is visualized as nuclear staining in dermal papillae of some follicles. FIG. 5C shows, at P25, that TRPS1 expression is visualized as nuclear staining in dermal papilla, and non-nuclear, punctate staining in epithelial cells just above dermal papilla. FIG. 5D shows, at P30, that TRPS1 staining is visualized as nuclear expression in dermal papilla and hair shaft cortex cells. The dotted line indicates basement membrane.

FIG. 6A-D show images of hematoxylin and eosin (H&E) stained sections of wild-type (TRPS1+/?) TRPS1 (FIGS. 6A, B) and knockout (TRPS1^{Δgt/Δgt}) TRPS1 (FIGS. 6C, D) mouse tissues. FIG. 6A shows a back-skin section of a wild-type mouse showing normal histology. FIG. 6B shows a whisker pad section of a wild-type mouse showing normal histology i.e., presence of normal vibrissa follicles. FIG. 6C shows a back-skin section of a TRPS1 knockout mouse showing a reduced number of developmentally delayed hair follicles. FIG. 6D shows a whisker pad section of a TRPS1 knockout mouse showing abnormal histology i.e. a reduced number of developmentally delayed hair follicles.

FIG. 7A-B show validation of potential target expression differences between e12.5 wild-type and TRPS1^{Δgt/Δgt} in the whisker pad. FIG. 7A shows sqRT-PCT validation of Prdm1, Sox18, Dkk4, Dspg3, Decorin and Lumican expression differences, which confirm the changes observed in the microarray. FIG. 7B shows RT-PCR validation of Prdm1, Sox18 and Dkk4 expression differences.

FIG. 8A-F show images of TRPS1 expression and its potential molecular targets in different compartments of a developing mouse embryo. FIG. 8A (Prdm1; e17.0) and FIG. 8B (TRPS1; P1) show overlapping expression in the dermal papilla. FIG. 8C (Sox18; e14.0) and FIG. 8D (TRPS1; e16.5) show overlapping expression in the mesenchyme surrounding the hair follicle. FIG. 8E (Dkk4; e14.0-14.5) and FIG. 8F (TRPS1; e14.0) show overlapping expression in the epidermis. The dotted line indicates basement membrane. FIG. 8A is adapted from Chang and Calame, 2002, Mech. Dev., 117:305-309; FIG. 8C is adapted from Pennisi et al., 2000, Nat. Genet. 24:434-437.

FIG. 9A-B show differences in Prdm1 expression in P1 TRPS1^{+/+} versus TRPS1^{-/-} whisker pads as detected by immunohistochemistry. FIG. 9A shows that Prdm1 is expressed in the granular layer of the epidermis in a TRPS1^{+/+} whisker pad. FIG. 9B shows that Prdm1 expression is significantly reduced in the granular layer in a TRPS1^{-/-} whisker pad.

FIG. 10A-D show differences in Dkk4 expression in P1 TRPS1^{+/+} versus TRPS1^{-/-} whisker pads as detected by immunohistochemistry. FIG. 10A shows that Dkk4 is expressed in the epithelial compartment of the skin and pelage follicle. FIG. 10B shows that Dkk4 expression is reduced in the hair shaft cuticle in TRPS1^{-/-} whisker pad. FIG. 10C shows that Dkk4 is expressed in the epithelial compartment of the skin and vibrissae follicles. FIG. 10D shows that Dkk4 is present only in the epidermis, as the mutant mice lack vibrissae follicles.

FIG. 11 shows a schematic of gradients of TRPS1 expression in the hair follicles of Ver-mTRPS1 mice. The versican promoter will drive expression of TRPS1 to the mesenchyme-derived dermal papilla. Adapted from Kishimoto et al., 1999, Proc. Natl. Acad. Sci. U.S.A. 96(13):7336-7341.

FIG. 12 shows a schematic of a Ver-myc-mTRPS1 transgenic construct. The versican promoter drives expression of a myc-mTRPS1 fusion protein to the mesenchymal compartment of the hair follicle.

FIG. 13 shows the DNA sequence of human TRPS1 mRNA (SEQ ID NO:1). The TRPS1 coding region is residues 639-4481 of SEQ ID NO:1.

FIG. 14 shows the amino acid sequence of the human TRPS1 protein (SEQ ID NO:2).

### DETAILED DESCRIPTION OF THE INVENTION

For clarity of presentation, and not by way of limitation, the detailed description of the invention is divided into the following subsections:

(i) transgenic animals;

(ii) assay systems;

(iii) methods of decreasing TRPS1 in a subject;

(iv) methods of directly increasing TRPS1 in a subject;

(v) methods of indirectly increasing TRPS1 in a subject;

(vi) methods of inhibiting hair growth in a subject; and

(vii) methods of promoting hair growth in a subject.

The present invention may be applied to a human or a non-human subject. Nonlimiting examples of non-human subjects that may benefit from the invention include domesticated farm, companion or laboratory animals, especially mammals, (such as cats, dogs, rabbits, ferrets, guinea pigs, rats, mice and hamsters) as well as animals that are used in the wool (e.g., sheep, alpaca, llama) or fur industries.

As used herein, the term TRPS1, without more, can refer to the TRPS1 gene, TRPS1 cDNA, TRPS1 mRNA, TRPS1 protein, or a combination thereof. The present invention encompasses TRPS1 nucleic acids and proteins of diverse species.

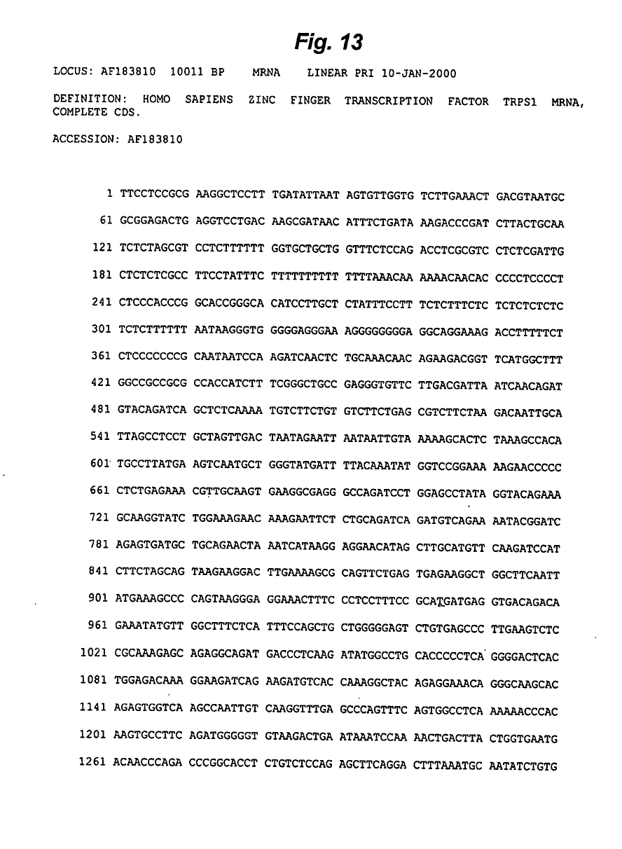

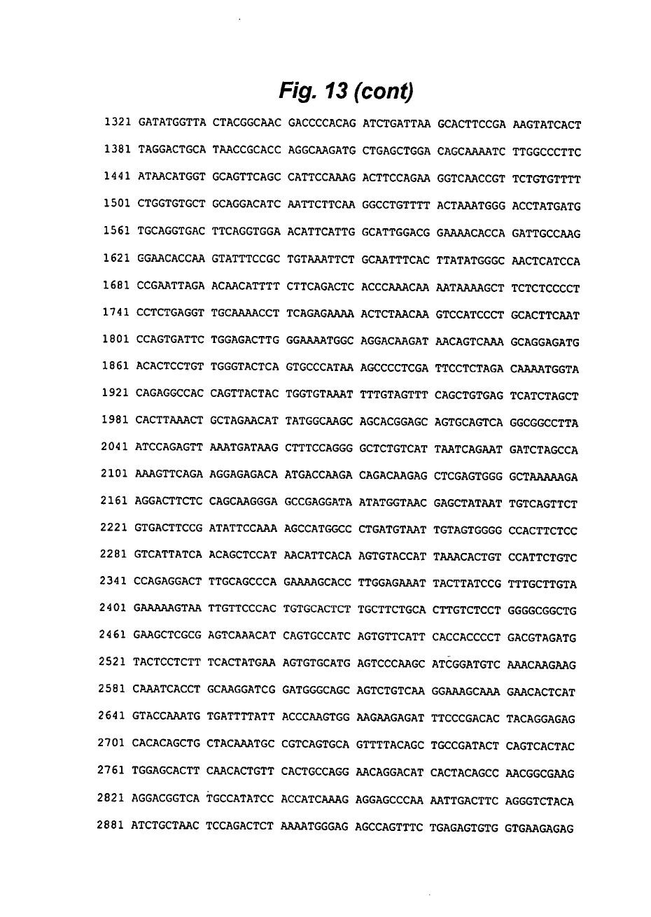

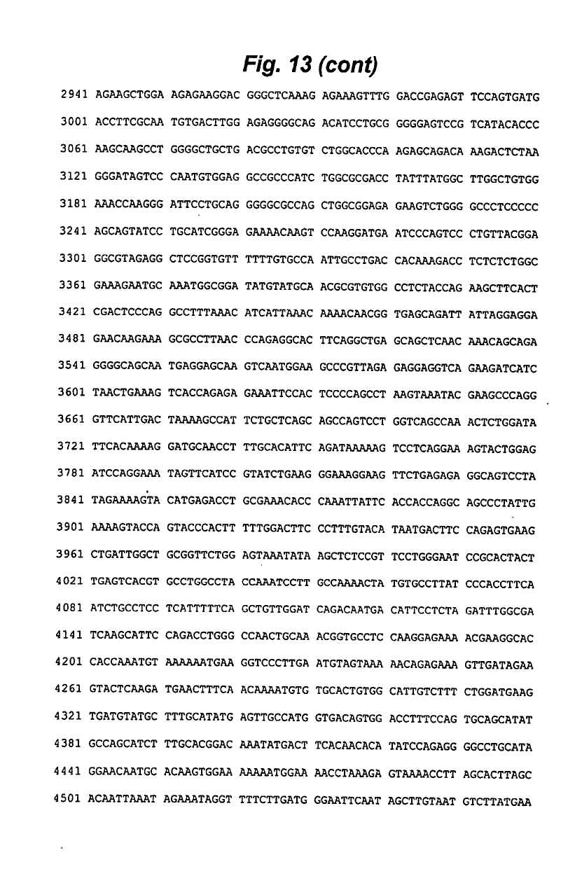

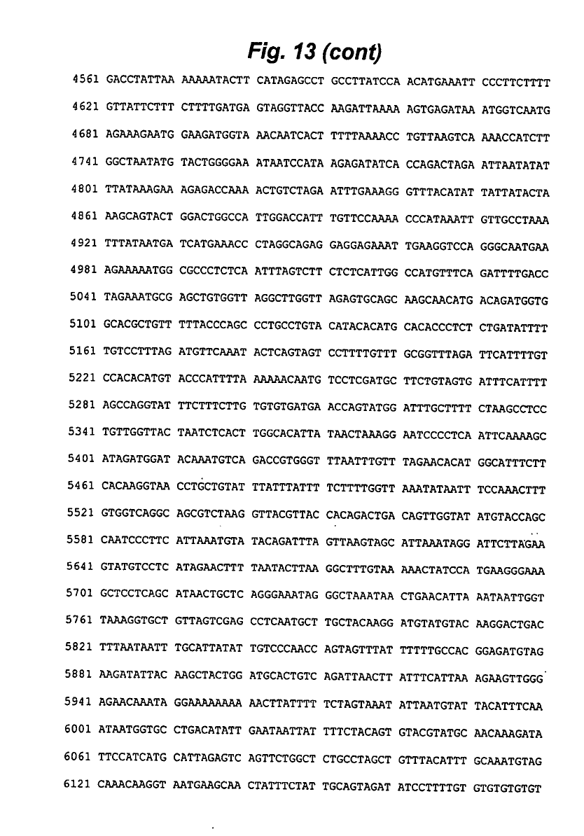

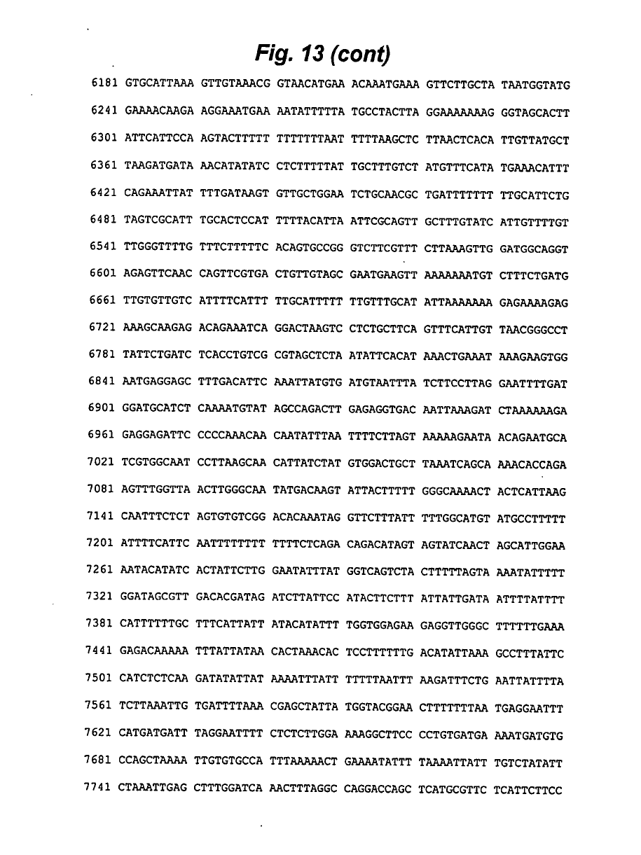

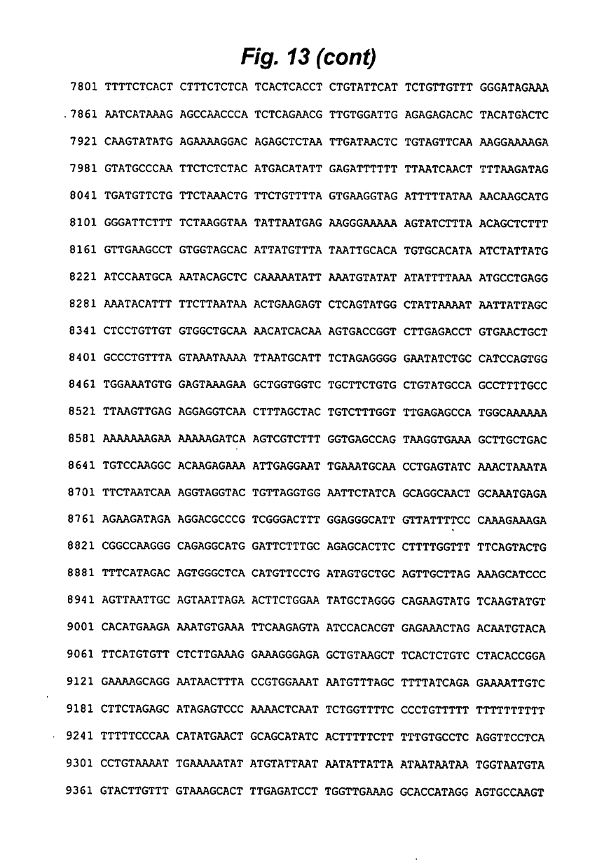

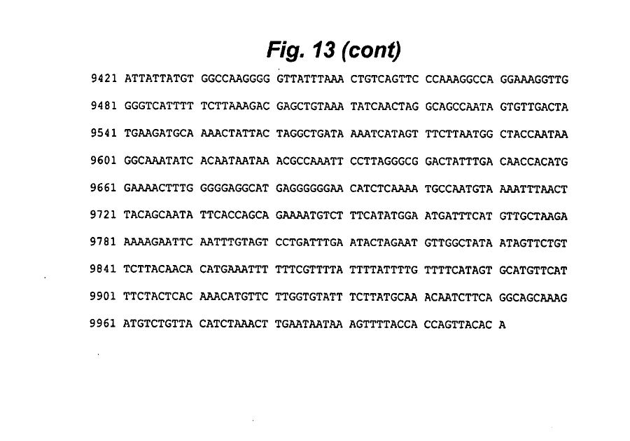

In particular nonlimiting embodiments, the present invention relates to a human TRPS1 nucleic acid having a sequence set forth in SEQ ID NO:1 and FIG. 13 with GenBank Accession numbers AF183810, NM_{—}014112 or AX578066, as well as to nucleic acids at least about 12, 15, 20, 25, 50, 100, 200, 500, 1000, 1500, 2000, 2500, 3000, 3500, 4000, 4500, 5000, 5500, 6000, 6500, 7000, 7500, 8000, 8500, 9000, 9500, 10000 nucleotides in length that either (i) are at least about 85, 90, or 95 percent homologous to the coding region of the TRPS1 gene of SEQ ID NO:1, residues 639-4481 of SEQ ID NO:1, where said coding region has SEQ ID NO:3; or its complement (as determined using standard software such as BLAST or FASTA) and/or (ii) hybridize to a comparable length of nucleic acid having a sequence as set forth in SEQ ID NO:3 or its complement under stringent conditions, defined as e.g., hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/0.1 percent SDS at 68° C. (Ausubel et al., 1989, Current Protocols in Molecular Biology, Vol. I, Green Publ. Assoc., Inc., and John Wiley & Sons, Inc., New York, at p. 2.10.3).

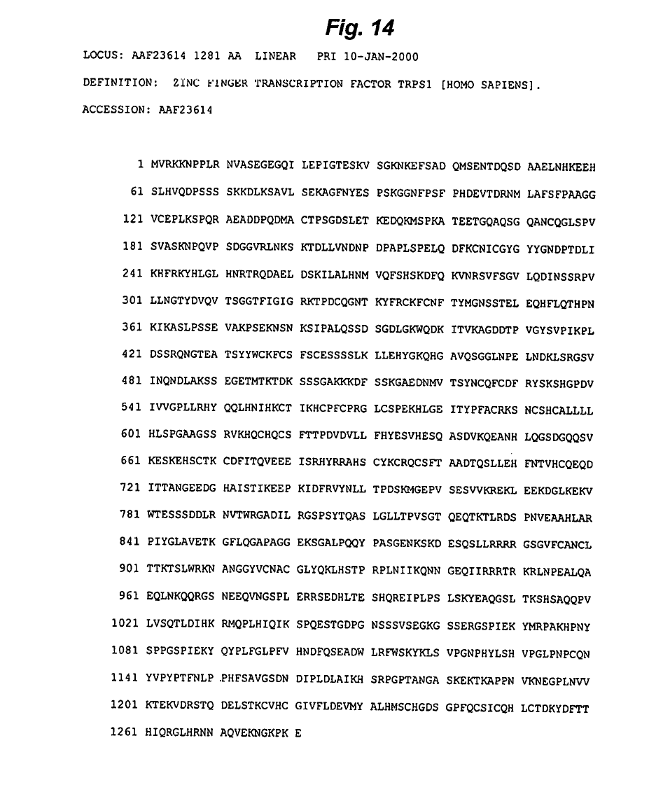

The present invention further provides for nucleic acids encoding a TRPS1 protein having a sequence as set forth in SEQ ID NO:2, FIG. 14. In other nonlimiting embodiments, the present invention relates to a non-human TRPS1 nucleic acids such as Mus musculus atypical GATA protein TRPS1 (GenBank accession numbers AF346836, BC037058, BC049857), Pan troglodytes zinc finger transcription factor TRPS1 (GenBank accession number XM_{—}528218), Gallus gallus similar to zinc finger transcription factor TRPS1 (GenBank accession numbers XM_{—}418402, XM_{—}418403), Bos taurus similar to zinc finger transcription factor TRPS1 (GenBank accession numbers NM_{—}450339, NW_{—}620498, XM_{—}608853, XM_{—}597092, XM_{—}617828, XM_{—}595745), and Rattus norvegicus similar to zinc finger transcription factor TRPS1 (GenBank accession number XM_{—}235264). Nucleic acids of the invention may be DNA, RNA, or cDNA, and may be single or double stranded, and include, within their scope, siRNA, antisense oligonucleotide, ribozyme and deoxyribozyme molecules.

The present invention further provides for TRPS1 proteins, defined as proteins encoded by any of the TRPS1 nucleic acids of the preceding paragraph. In specific, nonlimiting embodiments, the invention relates to a human TRPS1 protein having a sequence as set forth in SEQ ID NO:2 and FIG. 14 with GenBank accession numbers AAF23614, NP_{—}054831, Q925H1 or Q9UHF7 as well as to proteins or peptides at least about 12, 15, 20, 25, 50, 100, 150, 200, 250, 300, 350, 400, 450, 500, 550, 600, 650, 700, 750, 800, 850, 900, 950, 1000, 1050, 1100, 1150, 1200 and 1250 amino acid residues in length that are at least about 85, 90, or 95 percent homologous to a comparable length of sequence of SEQ ID NO:2 (as determined using standard software such as BLAST or FASTA). The present invention also encompasses non-human TRPS1 proteins such as Mus musculus atypical GATA protein TRPS1 (GenBank accession numbers AAK39508, AAH49875, AAH37058, AAH83110, NP_{—}114389, Q925H1), Xenopus laevis atypical GATA protein TRPS1 (GenBank accession numbers AAK39509, AAK39510), Gallus gallus predicted atypical GATA protein TRPS1 (GenBank accession number XP_{—}418402, XP_{—}418403), Pan troglodytes predicted atypical GATA protein TRPS1 (GenBank accession number XP_{—}528218), Canis familiaris predicted atypical GATA protein TRPS1 (GenBank accession number XP_{—}539139), Bos taurus predicted atypical GATA protein TRPS1, partial cDNA (GenBank accession number XP_{—}608853, XP_{—}597092, XP_{—}617828, XP_{—}595745, XP_{—}604931), Xenopus laevus atypical GATA protein TRPS1, partial cDNA (GenBank accession number Q90ZS6), and Rattus norvegicus similar to TRPS1 protein (GenBank accession number XP_{—}235264), and nucleic acids encoding said proteins.

### Transgenic Animals

The present invention provides for transgenic animals that contain a transgene that results in altered expression levels of TRPS1. In one set of nonlimiting embodiments, the transgene is a TRPS1 nucleic acid in antisense orientation, operably linked to a promoter element which may optionally be selectively or specifically active in hair follicle cells or be inducible. In yet another set of nonlimiting embodiments, the transgenic animal is a TRPS1 “knock-out” animal, in which the expression of at least one TRPS1 gene is inhibited by the introduction of foreign DNA in the TRPS1 gene or the chromosomal region containing the TRPS1 gene.

Nonlimiting examples of promoter elements that are specifically or selectively expressed in hair follicle cells include the versican promoter (Naso et al., 1994, J. Biol. Chem. 269(52):32999-33008; Kishimoto et al., 1999, Proc. Natl. Acad. Sci. U.S.A. 96(13):7336-7341); the fibroblast growth factor 18 promoter (Kawano et al., 2005, J. Invest. Dermatol. 124(5):877-885; Shimokawa et al., 2003, Cancer Res. 63:6116-6120); the osteopontin promoter (Yu et al., 2001, J. Invest. Dermatol. 117(6):1554-1558; Wang et al., 2000, Oncogene 19(50):5801-5809; Tezuka et al., 1996, J. Biol. Chem. 271(37):22713-22717); and the prolactin promoter (Foitzik et al., 2003, Am. J. Pathol. 162(5):1611-1621; Takasuka et al., 1998, Endocrinol. 139(3):136101368; Maurer et al., 1989, J. Biol. Chem. 264(12):6870-6873).

Trangenic animals which may be produced according to the invention include, but are not limited to, transgenic mice, rats, goats, sheep, cats, and pigs. Standard techniques in the construction and analysis of transgenic animals may be performed as described in manuals such as “Manipulating the mouse embryo, A laboratory manual” 3rd Edition, 2003 (Nagy et al. Eds., pp. 289-358; Cold Spring Harbor Laboratory Press, Cold Spring Harbor N.Y.). Transgenic animals may also be constructed by microinjection or electroporation of embryonic stem cells. The procedure to construct a transgenic line from an ES cell is also well known in the art and may be performed for example as described in Nagy et al., 2003, supra.

Targeted disruption of a gene to generate a null or mutated allele is usually accomplished by insertion of a selectable marker (usually neomycin) into a gene causing disruption of splicing, promoter function, or reading frame, with or without deletion of some of the gene. In a particular nonlimiting embodiment, a TRPS1 “knock-out” animal is one in which the nucleic acid sequence encoding the GATA domain of the TRPS1 protein has been disrupted. Mice homozygous for a deletion of the GATA domain in TRPS1 (TRPS1^{Δgt/Δgt}) have been found to die within hours after birth. In a specific, non-limiting embodiment, the invention provides for grafting skin of a TRPS1^{Δgt/Δgt} mouse into a nude mouse to provide a model for TRPS1 knockout skin/hair while circumventing the neonatal lethality issue.

Incorporation of the altered gene into the mouse genome depends upon replacement of the endogenous gene by homologous recombination through both of the arms of the altered gene into one allele of genomic DNA. Methods to prepare a mouse line with a targeted disruption or knockout of a specific gene is well known to the art and may for example be performed by routine techniques described in Nagy et al., 2003, supra.

Conditional knockout mice may be prepared using the Cre-loxP System: When global removal of a gene of interest using conventional knockout methods results in embryonic lethality, a conditional knockout line of mice may be produced where tissue-specific deletions can still be studied in vivo. To knock out a gene in a specific tissue, an important exon of the targeted gene is subcloned in between two loxP sites (ie., clone loxP elements in each of the introns flanking the exon to be deleted). Homologous recombinants are generated by a homologous recombination replacement event as with conventional targetings described above. In such animals the gene will retain normal function in the ES cells and in the parental chimeras and agouti heterozygotes and homozygotes, thus allowing the mice to survive and breed. After germline transmission has occurred and the mice are bred to homozygosity, the “floxed” mice may be bred to a tissue-specific Cre transgenic mouse, and in the resulting offspring, the exon will be removed only in that tissue. Thus the effect of ablation a gene activity may be studied in a cell, tissue or organ specific manner and the problem or embryonal lethality due to loss of the targeted gene is overcome. Conditional knockout mice may be constructed by methods well known to the art and may for example be performed by routine techniques described in Nagy et al., 2003, supra.

### Assay Systems

The present invention provides for assay systems that may be used to identify agents that decrease or increase TRPS1 expression. The assay systems may utilize isolated cells, multicellular structures, or organisms, including, but not limited to, the transgenic animals described above.

In a first set of nonlimiting embodiments, the present invention evaluates the effects of test agents on endogenous TRPS1 expression. Accordingly, the present invention provides for a method of identifying an agent that inhibits hair growth, comprising administering, to a test cell or a test organism, a test agent, and determining whether the test agent induces a decrease in the level of TRPS1 expression, as measured by mRNA or protein, which may be performed using standard techniques including, but not limited to, PCR, Northern blot, or Western blot. The change in TRPS1 level may be measured relative to the TRPS1 level in a control cell or organism not exposed not test agent. A test agent that decreases TRPS1 levels is likely to inhibit hair growth, and may optionally be administered to a test subject to determine whether hair growth is inhibited in the test subject.

In a second set of non-limiting embodiments, the present invention provides for a method of identifying an agent that promotes hair growth, comprising administering, to a test cell or a test organism, a test agent, and determining whether the test agent induces an increase in the level of TRPS1 expression, as measured by mRNA or protein, which may be performed using standard techniques including, but not limited to, PCR, Northern blot, or Western blot. The change in TRPS1 level may be measured relative to the TRPS1 level in a control cell or organism not exposed not test agent. A test agent that increases TRPS1 levels is likely to promote hair growth, and may optionally be administered to a test subject to determine whether hair growth is promoted in the test subject.

In a third set of nonlimiting embodiments, the present invention evaluates the effects of test agents on a reporter construct comprising a TRPS1 binding element linked to a reporter gene. Such reporter constructs are known in the art (see, for example, van den Bemd et al., 2003, Biochem. Biophys. Res. Commun. 312(3):578-584), and may be used to monitor levels of TRPS1 expression. In a nonlimiting set of embodiments, the present invention provides for a method of identifying a test agent that modulates TRPS1 expression or activity, comprising administering the test agent to a test cell or test organism containing a TRPS1-binding element/reporter gene construct, and then determining whether the test agent decreases or increases the level of reporter gene product, e.g., relative to the level of reporter gene product in a control cell or organism not exposed to the test agent. Specific nonlimiting examples include the use of luciferase or green fluorescent protein in assay systems utilizing isolated cells, and the use of green fluorescent protein in assay systems utilizing whole organisms.

Transgenic animals in which TRPS1 is overexpressed may be used in analogous assay systems to identify agents which inhibit TRPS1 expression or activity. “Activity,” as used in this paragraph, refers to the ability of TRPS1 to promote hair growth, where an agent is envisioned which may not increase expression of TRPS1 but which may render the endogenous TRPS1 protein more potent in promoting hair growth.

### Methods of Decreasing TRPS1 in a Subject

The present invention further provides for methods of decreasing TRPS1 expression in a subject. Such a decrease may be effected, for example, by administering, to a subject in need of such treatment, either an agent identified as decreasing TRPS1 mRNA and/or protein, using an assay system as set forth above, or by administering an effective amount of an siRNA, antisense oligonucleotide, or catalytic nucleic acid (e.g., ribozyme or deoxyribozyme) directed at the nucleic acid sequence of the endogenous TRPS1 gene. In alternate embodiments, the siRNA, antisense oligonucleotide or catalytic nucleic is directed at the coding region of the TRPS1 gene, residues 639-4481 of SEQ ID NO:1, where said coding region has SEQ ID NO:3.

Catalytic Nucleic Acids

In nonlimiting embodiments of the invention, expression of the TRPS1 gene may be decreased using a catalytic nucleic acid molecule, such as a ribozyme, i.e., an RNA molecule with catalytic activity. See, e.g., Cech, 1987, Science 236:1532-1539; Cech, 1990, Annu. Rev. Biochem. 59:543-568; Cech, 1992, Curr. Opin. Struct. Biol. 2:605-609; Couture and Stinchcomb, 1996, Trends Genet. 12:510-515. In particular nonlimiting embodiments, the catalytic nucleic acid molecule is between about 13 and 500 nucleotides in length. In alternate embodiments, the catalytic nucleic acid molecule is between about 10 and 500 nucleotides, or between about 13 and 200 nucleotides. In specific nonlimiting embodiments, the catalytic nucleic acid molecule is between 13 and 100 nucleotides, or between about 13 and 50 nucleotides.

Ribozymes can be used to inhibit TRPS1 gene function by cleaving an RNA sequence, as is known in the art (e.g., Haseloff et al., U.S. Pat. No. 5,641,673). The 5′ untranslated, 3′ untranslated or coding sequence of the TRPS1 gene may be used to generate a ribozyme which will specifically bind to mRNA transcribed from the TRPS1 gene. Methods of designing and constructing ribozymes which can cleave other RNA molecules in trans in a highly sequence specific manner have been developed and described in the art (see Haseloff et al., 1988, Nature 334:585-591).

In other nonlimiting embodiments, the catalytic nucleic acid molecules of the instant invention are DNA oligonucleotides that have a structure similar to the hammerhead ribozyme (Santoro and Joyce, 1997, Proc. Natl. Acad. Sci. USA, 94:4262-6). These molecules are known as “deoxyribozymes” and “DNAzymes” and are virtually DNA equivalents of the hammerhead ribozymes. They consist of a 15-bp catalytic core and two sequence-specific arms with a typical length of 5-13 basepairs each (Santoro and Joyce, 1998, Biochemistry, 37:13330-42.). Deoxyribozymes have more lenient consensus cleavage site requirements than hammerhead ribozymes, and are less likely to degrade when used for in vivo applications. The most widely used type of these novel catalytic molecules is known as the “10-23” deoxyribozyme, whose designation originates from the numbering used by its developers (Santoro and Joyce, 1997, supra). Because of their considerable advantages, deoxyribozymes have been used in a wide spectrum of in vitro and in vivo applications (Cairns et al., 2000, Nucleic Acids Res., 28:E9; Santiago et al., 1999, Nat. Med., 5:1438).

TRPS1 Antisense Oligonucleotides

In other nonlimiting embodiments of the invention, expression of the TRPS1 gene may be inhibited using an antisense oligonucleotide sequence. As used herein, a TRPS1 antisense oligonucleotide is an antisense sequence complementary to at least a portion of the 5′ untranslated, 3′ untranslated or coding sequence of the TRPS1 gene. Preferably, the antisense oligonucleotide is an antisense sequence complementarity to at least a portion of the coding sequence of the TRPS1 gene, wherein said coding sequence has SEQ ID NO:3. Preferably, the antisense oligonucleotide sequence is at least six nucleotides in length, but can be up to about 50 nucleotides long. In nonlimiting embodiments, the antisense oligonucleotide sequence is between about 10 and 35 nucleotides, or between about 10 and 25 nucleotides. In a particular nonlimiting embodiment, the antisense oligonucleotide sequence is between about 15 and 25 nucleotides. Longer sequences can also be used. The antisense oligonucleotides of the invention may be DNA, RNA, or any modifications or combinations thereof. As an example of the modifications that the oligonucleotides may contain, inter-nucleotide linkages other than phosphodiester bonds, such as phosphorothioate, methylphosphonate, methylphosphodiester, phosphorodithioate, phosphoramidate, phosphotriester, or phosphate ester linkages (Uhlman et al., 1990, Chem. Rev. 90(4):544-584; Tidd, 1990, Anticancer Res. 10(5A):1169-1182), may be present in the oligonucleotides, resulting in their increased stability. Oligonucleotide stability may also be increased by incorporating 3′-deoxythymidine or 2′-substituted nucleotides (substituted with, e.g., alkyl groups) into the oligonucleotides during synthesis, by providing the oligonucleotides as phenylisourea derivatives, or by having other molecules, such as aminoacridine or poly-lysine, linked to the 3′ ends of the oligonucleotides (see, e.g., Tidd, 1990, supra). Modifications of the RNA and/or DNA nucleotides comprising the oligonucleotides of the invention may be present throughout the oligonucleotide, or in selected regions of the oligonucleotide, e.g., the 5′ and/or 3′ ends. The antisense oligonucleotides may also be modified so as to increase their ability to penetrate the target tissue by, e.g., coupling the oligonucleotides to lipophilic compounds. The antisense oligonucleotides of the invention can be made by any method known in the art, including standard chemical synthesis, ligation of constituent oligonucleotides, and transcription of DNA encoding the oligonucleotides, as described below. Precise complementarity is not required for successful duplex formation between an antisense molecule and the complementary coding sequence of the TRPS1 gene. Antisense molecules which comprise, for example, 2, 3, 4, or 5 or more stretches of contiguous nucleotides which are precisely complementary to a portion of a coding sequence of the TRPS1 gene, each separated by a stretch of contiguous nucleotides which are not complementary to adjacent coding sequences, can provide targeting specificity for mRNA of the TRPS1 gene. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and the TRPS1 gene untranslated or coding sequence.

RNAi or siRNA

In further nonlimiting embodiments of the invention, dsRNA-mediated interference (RNAi or siRNA), which is well known in the fields of molecular biology, may be used to inhibit the expression of the TRPS1 gene (see, for example, Hunter, 1999, Curr. Biol. 9:R440-442; Hamilton et al., 1999, Science 286:950-952; Ding, 2000, Curr. Opin. Biotechnol., 11:152-156). siRNA typically comprises a polynucleotide sequence identical or homologous to a target gene (or fragment thereof) linked directly, or indirectly, to a polynucleotide sequence complementary to the sequence of the target gene (or fragment thereof). In a nonlimiting example, a RNAi or siRNA molecule should be between about 6 and 50 nucleotides. In particular nonlimiting examples, the RNAi or siRNA molecule is between about 10 and 35 nucleotides, or between about 10 and 25, or between about 15 and 25.

The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of siRNA sufficiently as to limit the effects of steric hindrance and allow for the formation of dsRNA molecules and should not hybridize with sequences within the hybridizing portions of the dsRNA molecule. Accordingly, one method for inhibiting hair growth comprises the use of (siRNA) comprising polynucleotide sequences identical or homologous to the TRPS1 gene.

RNA containing a nucleotide sequence identical to a fragment of the target gene is preferred for inhibition; however, RNA sequences with insertions, deletions, and point mutations relative to the target sequence can also be used for inhibition. As described above for antisense molecules, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Preferably, siRNA is targeted to a polynucleotide sequence of the TRPS1 gene. Preferred siRNA molecules of the instant invention are highly homologous or identical to the polynucleotides encoding the TRPS1 gene. The homology may be greater than 70%, preferably greater than 80%, more preferably greater than 90% and is most preferably greater than 95%.

Ribozymes, antisense polynucleotides, and siRNA molecules may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus.

RNA may also be chemically or enzymatically synthesized by manual or automated reactions. The RNA may be synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Ribozymes, antisense molecules, and siRNA can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells. Mechanical methods, such as microinjection, liposome-mediated transfection, electroporation, or calcium phosphate precipitation, can be used to introduce such DNA constructs into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

Administration of one or more agent that decreases TRPS1 may be achieved by methods known in the art, including topical, intracutaneous, intravenous, or oral administration. In preferred nonlimiting embodiments of the invention, administration is targeted to hair follicle cells to be treated. The one or more agent may be comprised in a composition together with suitable pharmaceutical carriers. The agent may optionally be comprised in a microstructure such as a liposome or microsphere. A composition comprising the agent may further comprise a permeability-enhancing agent such as such as dimethylsulfoxide, lipofectamine, oligofectamine, nanoparticles, and/or cyclofectin.

One or more agent may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, nonlimiting embodiment of the invention, the interval between treatments or treatment periods may be the average length of time, in the subject to be treated, for a hair follicle to pass through one cycle.

According to the invention, one or more agent that inhibits TRPS1 expression may be administered ex vivo to a hair follicle cell, which may be an isolated cell or a cell in a hair follicle and/or dermal papilla in culture. Methods of harvesting hair follicles and dermal papillae, and maintaining them in culture, are known in the art.

### Methods of Directly Increasing TRPS1 in a Subject

The present invention provides for methods of directly increasing TRPS1 in a cell of a subject comprising introducing, into said cell, a nucleic acid encoding TRPS1 in expressible form or introducing, into said cell, TRPS1 protein. Preferably, but not by way of limitation, the cell is a hair follicle cell, where the hair follicle cell may be comprised in the skin of a subject or may be an isolated cell or may be part of an isolated multicellular structure (e.g., an isolated follicle, a dermal papilla or a placode). In specific, non-limiting embodiments, the hair follicle cell is a dermal papilla cell.

In a nonlimiting example, the present invention provides for a method for increasing TRPS1 expression in a cell by introducing, into a cell, comprising a nucleic acid selected from the group consisting of (i) SEQ ID NO:3; (ii) a sequence 90 percent homologous to SEQ ID NO:3; and (iii) a sequence which encodes a TRPS1 protein with amino acid sequence SEQ ID NO:2. The present invention further provides for a composition used to increase TRPS1 expression in a cell, comprising a nucleic acid selected from the group consisting of (i) SEQ ID NO:3; (ii) a sequence 90 percent homologous to SEQ ID NO:3; and (iii) a sequence which encodes a TRPS1 protein with amino acid sequence SEQ ID NO:2.

Where TRPS1 expression is increased by introducing a TRPS1 encoding nucleic acid into a cell, the nucleic acid may be operably linked to a promoter element, and optionally to other elements that aid in transcription and/or translation. The promoter element may be selectively or specifically active in a hair follicle cell, or may be expressed in diverse tissues. Alternatively, the promoter element may be inducible. Non-limiting examples of promoter elements that are specifically or selectively expressed in hair follicle cells include the versican promoter (Naso et al., 1994, supra; Kishimoto et al., 1999, supra); the fibroblast growth factor 18 promoter (Kawano et al., 2005, supra; Shimokawa et al., 2003, supra); the osteopontin promoter (Yu et al., 2001, supra; Wang et al., 2000, supra; Tezuka et al., 1996, supra); and the prolactin promoter (Foitzik et al., 2003, supra; Takasuka et al., 1998, supra; Maurer et al., 1989, supra). Non-limiting examples of promoter elements that are inducible include tetracycline inducible promoters and metallothionine inducible promoters. Other non-limiting examples of promoters that may be used according to the invention include the cytomegalovirus immediate early promoter.

A TRPS1-encoding nucleic acid, in expressible form (e.g., operably linked to a promoter element), may optionally be comprised in a vector molecule. Suitable expression vectors include virus-based vectors and non-virus based DNA or RNA delivery systems. Examples of appropriate virus-based gene transfer vectors include, but are not limited to, pCEP4 and pREP4 vectors from Invitrogen, and, more generally, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6:113-138; Connelly, 1999, Curr. Opin. Mol. Ther. 1:565-572; Stratford-Perricaudet, 1990, Human Gene Ther. 1:241-256; Rosenfeld, 1991, Science 252:431-434; Wang et al., 1991, Adv. Exp. Med Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce cells and that can accommodate the TRPS-1 encoding nucleic acid and sequences necessary and/or desirable for its expression.

Where TRPS1 protein levels in a cell are to be increased by direct administration of TRPS1 protein to a cell, the TRPS1 protein is preferably comprised in a structure that facilitates uptake by a cell, preferably a hair follicle cell, more preferably a dermal papilla cell. For example, TRPS1 protein may be comprised in a liposome, microsphere or microbead. TRPS1 protein, optionally contained in a liposome, microsphere, or microbead, may be introduced into hair follicles in vivo via topical application or local injection, or ex vivo (see below).

Administration of TRPS1-encoding nucleic acid (optionally contained in a vector molecule) or TRSP1 protein may be achieved by methods known in the art, including topical, intracutaneous, intravenous, or oral administration. In preferred non-limiting embodiments of the invention, administration is targeted to hair follicle cells to be treated. In one set of non-limiting embodiments, targeting may be achieved by route and/or site of administration, for example, topical application or injection into an area where promotion of hair growth is desired. In another set of non-limiting embodiments, targeting may be achieved by promoter selection, for example, operably linking a TRPS1 nucleic acid to a promoter selectively active in hair follicle cells or an inducible promoter, where the inducing agent is selectively applied to hair follicle cells (e.g., topically or systemically administered TRPS1 nucleic acid is operably linked to a tetracycline-inducible promoter, and promoter activity is induced by systemic administration or topical application of tetracycline). TRPS1 nucleic acid may also be introduced into hair follicle cells ex vivo (see below).

One or more agent that directly increases TRPS1 may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, non-limiting embodiment of the invention, the interval between treatments or treatment periods may be the average length of time, in the subject to be treated, for a hair follicle to pass through one growth cycle.

The present invention provides for the introduction of TRPS1 nucleic acid operably linked to a promoter element and optionally contained in a vector, and/or TRPS1 protein, into hair follicles or components thereof maintained in culture (i.e., ex vivo TRPS1 gene and/or protein delivery). Hair follicles/dermal papillae may be harvested from a human subject or a non-human animal by methods known in the art or by improvements thereof, as they become available. Non-limiting methods of harvesting hair follicles include harvesting a “plug” of hair bearing tissue removed by an appropriate surgical instrument such as a punch e.g. a standard 4 mm punch. Alternatively, a multiple-strip method of harvesting hair follicles may be accomplished by passing a series of parallel scalpel blades (the multi-bladed knife) through the donor area. A more recent development utilizing single strip harvesting, by removal of a section of tissue either with two parallel blades forming a single long, thin strip or with a single blade producing a long, thin oval, may also be used. Surgery for harvesting hair follicles may be performed with or without the aid of a dissection microscope and encompasses both standard surgical as well as microsurgery or microdissection procedures. Hair follicles/dermal papillae may be maintained in culture, for example as described by Philpott et al., 1996 (Dermatol Clin., 14(4):595-607). TRPS1-encoding nucleic acid, as set forth above, may be introduced into the cells of the cultured hair follicles/dermal papillae using methods known in the art, such as transfection of an isolated DNA or RNA by liposomes or other chemically mediated methods, transduction by means of a replicating or non-replicating viral vector, electroporation, biolistic gene delivery, microporation (Bramson et al., 2003 Gene Ther. 10(3):251-260) etc. Gene delivery to a hair follicle may result in transient expression of the delivered gene which may require several applications for effective dosage. Alternatively, the gene may be delivered by a method which causes its permanent incorporation into the genome of the target cell population so as to constitutively express in the target cells and its progeny. Further, TRPS1 protein may be introduced into cells of cultured hair follicles/dermal papillae, for example, where the protein is contained in liposomes or microspheres or on microbeads which are internalized by hair follicle cells. In non-limiting embodiments, TRPS1 nucleic acid and/or protein may be introduced into dermal papilla cells in culture, whereby the period of inductivity of at least a portion of the cultured dermal papilla cells is prolonged, and/or the inductivity of at least a portion of the dermal papilla cells is enhanced or at least partially restored (where “inductivity” refers to the ability of dermal papilla cells to induce the formation of a hair follicle).

The foregoing methods of directly increasing levels of TRPS1 may be used to promote hair growth in a subject in need of such treatment. In non-limiting embodiments, such methods may be used to treat alopecia in a human subject, such as occurs, for example, in male pattern baldness. In other embodiments, such methods may be used to promote hair growth in a domesticated animal which is a laboratory animal, farm animal, pet, or which is used in the wool or fur industries.

The present invention likewise comprises compositions that may be used in the above methods, for example, compositions comprising TRPS1 nucleic acid operably linked to a promoter element, optionally contained in a vector, where the composition is a topical formulation or a pharmaceutical preparation suitable for injection; compositions comprising TRPS1 protein, optionally contained in a liposome, microsphere, or microbead, where the composition is a topical formulation or a pharmaceutical preparation suitable for injection. Topical formulations include creams, lotions, ointments, etc. Injectable formulations include but are not limited to saline solutions. A composition comprising TRPS1 nucleic acid and/or protein may further comprise a permeability-enhancing agent such as dimethylsulfoxide, lipofectamine, oligofectamine, nanoparticles, and/or cyclofectin. In still further embodiments, the present invention provides for hair follicle cells into which TRPS1 nucleic acid and/or protein has been introduced ex vivo.

### Methods of Indirectly Increasing TRPS1 in a Subject

The present invention further provides for methods which indirectly increase TRPS1 mRNA and/or protein. Such methods do not administer TRPS1-encoding nucleic acid or TRPS1 protein, but rather administer an agent which results in an increase in TRPS1 mRNA transcription, its translation into protein, and/or the half-life and/or functional activity of TRPS1 mRNA or TRPS1 protein. The agent may be, for example but not by way of limitation, a small molecule (e.g., a member of a library developed through combinatorial chemistry), a peptide, a protein, a nucleic acid, a lipid, or a carbohydrate.

As androgens inhibit expression of TRPS1, an agent that may be used to indirectly increase TRPS1 may be an agent that antagonizes androgen action. For example, such an agent may inhibit formation of active androgen, may inhibit binding of androgen to its receptor (e.g., in hair follicle cells), or may inhibit intracellular signaling by the androgen-bound receptor. Known androgen antagonists include flutamide, bicalutamide, vinclozolin, and 3,3′-diindolylmethane. Agents that inhibit formation of androgen include agents that inhibit 5α-reductase such as finasteride. In non-limiting embodiments of the invention, an agent that inhibits 5α-reductase expression or androgen receptor expression in a subject may be a siRNA, antisense oligonucleotide, or catalytic nucleic acid (e.g. ribozyme or deoxyribozyme) with at least a portion complementary to a mRNA encoding 5α-reductase or the androgen receptor, respectively. In specific, non-limiting embodiments of the invention, the androgen antagonist is not finasteride, flutamidine, bicalutamide, vinclozolin, or diindoylmethane.

In alternate embodiments, as RNF4 is a negative regulator of TRPS1 (Kaiser et al., 2003, J. Biol. Chem. 278(40):38780-38785), an agent that may be used to indirectly increase TRPS1 may be an agent that antagonizes RNF4. One non-limiting example of such an agent may be a siRNA, antisense oligonucleotide, or catalytic nucleic acid (e.g., ribozyme or deoxyribozyme) with at least a portion which is complementary to a mRNA encoding RNF4.

Other agents that may be used to indirectly increase TRPS1 in a cell, preferably a hair follicle cell, more preferably a dermal papilla cell, may be identified using the assay systems set forth herein.

Administration of one or more agent that indirectly increases TRPS1 (or a direct agent and one or more indirect agents) may be achieved by methods known in the art, including topical, intracutaneous, intravenous, or oral administration. In preferred non-limiting embodiments of the invention, administration is targeted to hair follicle cells to be treated. The one or more agent may be comprised in a composition together with suitable pharmaceutical carriers. The agent may optionally be comprised in a microstructure such as a liposome or microsphere. A composition comprising the agent(s) may further comprise a permeability-enhancing agent such as dimethylsulfoxide, lipofectamine, oligofectamine, nanoparticles, and/or cyclofectin.

One or more agent may be introduced into hair follicle cells ex vivo, for example as set forth in the preceding section.

One or more agent may be administered as a single application or as multiple applications over a period of time. Administration of agent may be, for example and not by way of limitation, once or twice daily, once or twice weekly, once a month, twice a month, once every two months, or once a year. Alternatively, the agent(s) may be administered for a treatment period, followed by a period of no treatment, followed by another treatment period, and this cycle may be repeated. In a specific, non-limiting embodiment of the invention, the interval between treatments or treatment periods may be the average length of time, in the subject to be treated, for a hair follicle to pass through one growth cycle.

The foregoing methods of indirectly increasing levels of TRPS1 by administering an effective amount of one or more agent may be used to promote hair growth in a subject in need of such treatment. In non-limiting embodiments, such methods may be used to treat alopecia in a human subject, such as occurs, for example, in male pattern baldness. In other embodiments, such methods may be used to promote hair growth in a domesticated animal which is a laboratory animal, farm animal, pet, or which is used in the wool or fur industries.

### Methods of Inhibiting Hair Growth in a Subject

The foregoing methods of decreasing levels of TRPS1 by administering an effective amount of one or more agent may be used to inhibit hair growth in a subject in need of such treatment. In nonlimiting embodiments, such methods may be used to treat hypertrichosis, or may be used as cosmetic depilatory agents. In other embodiments, such methods may be used to inhibit hair growth in a domesticated animal which is a laboratory animal, farm animal, pet, or which is used in the leather industry.

In a nonlimiting embodiment, the present invention further provides for methods for inhibiting hair growth in a subject. Such an inhibition of hair growth may be effected, for example, by administering, to a subject in need of such treatment, either an agent identified as decreasing TRPS1 mRNA and/or protein, using an assay system as set forth above, or by administering an effective amount of an siRNA, antisense oligonucleotide, or catalytic nucleic acid (e.g., ribozyme or deoxyribozyme), described supra, directed at TRPS1 or a downstream target of TRPS1. In a particular embodiment, the level of TRPS1 is decreased by introducing, into a cell of a subject, TRPS1 mRNA or protein.

In an alternate embodiment, the level and/or activity of TRPS1 is decreased by providing an agent that results in decreased expression of an endogenous TRPS1 gene, decreased functional activity of a TRPS1 protein, or decreased expression and/or activity of a target of a TRPS1 protein. In a particular embodiment, the agent is a catalytic nucleic acid, a siRNA or an antisense oligonucleotide directed to the endogenous TRPS1 gene. In an alternate embodiment, the agent is a catalytic nucleic acid, a siRNA or an antisense oligonucleotide directed to a target of TRPS1, such as Prdm1, Sox 18 or Dkk4, as nonlimiting examples. The characteristics of, and compositions for and methods of administering an effective amount of a catalytic nucleic acid, a siRNA and an antisense oligonucleotide used to inhibit downstream targets of TRPS1 are the same as those set forth for catalytic nucleic acids, siRNA, and antisense oligonucleotide used to inhibit expression of TRPS1, as described above.

Downstream targets of TRP1 include genes whose expression is decreased in TRPS1^{-/-} mice, and whose expression is increased in TRPS^{-/-} mice. Of interest, Prdm1, Sox18 and Dkk4 were detected among genes expressed at lower levels in the mutant animals. Sox18 and Prdm1 have been previously localized to the hair follicle dermis and contain 10 and 8 GATA-1 sites, respectively, in their upstream promoters. Dkk4 has recently been localized to the epidermis at sites of epithelial-mesenchymal interactions (Bazzi, unpublished data) and contains 5 GATA-1 sites within 2 kb upstream of the transcriptional start site.

Prdm1 is a Krüppel-type zinc finger protein responsible for terminal differentiation of B lymphocytes (Turner et al., 1994, Cell 77:297-306) and macrophage differentiation (Chang et al., 2000, Nat. Immunol. 1:169-176). Targeted disruption of the gene results in premature death at e10.5 (Vincent et al., 2005, Development 132:1315-1325). Prdm1 is expressed in the granular layer of the skin and the inner root sheath (IRS) precursors and dermal papilla of the hair follicle (Chang and Calame, 2002, Mech. Dev., 117:305-309).

Sox18 is an SRY-related transcription factor (Hosking et al., 1995, Nucleic Acids Res., 23:2626-2628) expressed in the underlying mesenchyme of hair follicles during mouse embryogenesis (Pennisi et al. 2000, Nat. Genet. 24:434-437). Mutations in Sox18 are responsible for the ragged (Ra) mouse phenotype, characterized by varying degrees of coat sparseness. Heterozygotes have short vibrissae and a thin, ragged coat, whereas homozygotes almost completely lack vibrissae and coat hairs (Carter and Phillips, 1954, J. Hered., 45:151-15). In humans, mutations in SOX18 result in hypotrichosis-lymphedema-telangiectasia syndrome, characterized by sparse scalp hair and the absence of eyebrows and eyelashes (Irrthum et al., 2003, Am. J. Hum. Genet., 72:1470-1478).

Dkk4 is a secreted Wnt antagonist that acts similar to Dkk1 in its ability to inhibit Wnt signaling (Mao and Niehrs, 2003, Gene 302:179-183). Dkk4 has recently been localized to the epidermis at sites of epithelial-mesenchymal interactions during mouse embryogenesis.

### Methods of Promoting Hair Growth in a Subject

The present invention provides for methods of promoting hair growth comprising increasing the level and/or activity of TRPS1 mRNA and/or protein in cells, preferably hair follicle cells, and more preferably dermal papilla cells, of a subject. The level of TRPS1 may be increased either directly, for example by introducing, into a hair follicle cell, TRPS1 mRNA or protein, or it may be increased indirectly, by providing an agent that results in increased expression of an endogenous TRPS1 gene, increased functional activity of TRPS1 protein, or increased expression of a target of TRPS1.

### EXAMPLE 1

### TRPS1 Expression

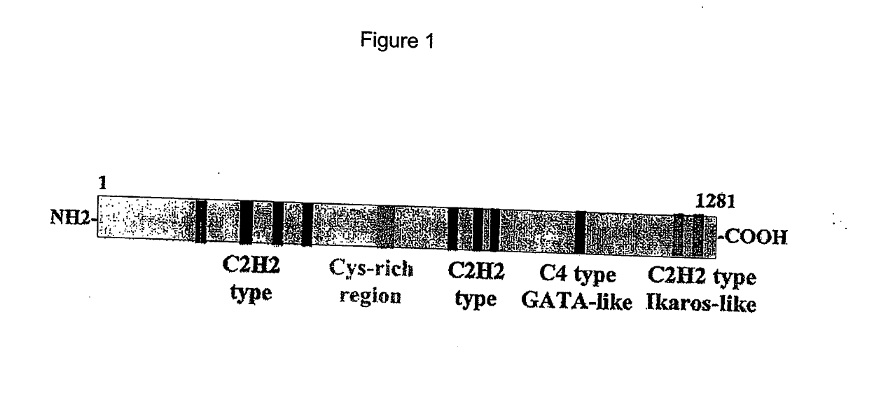

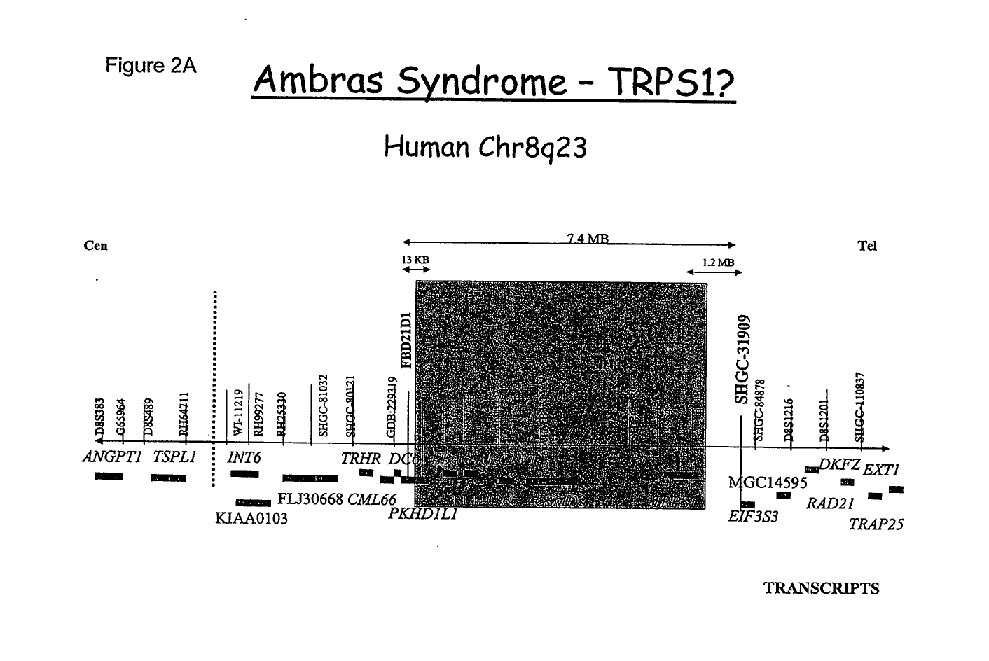

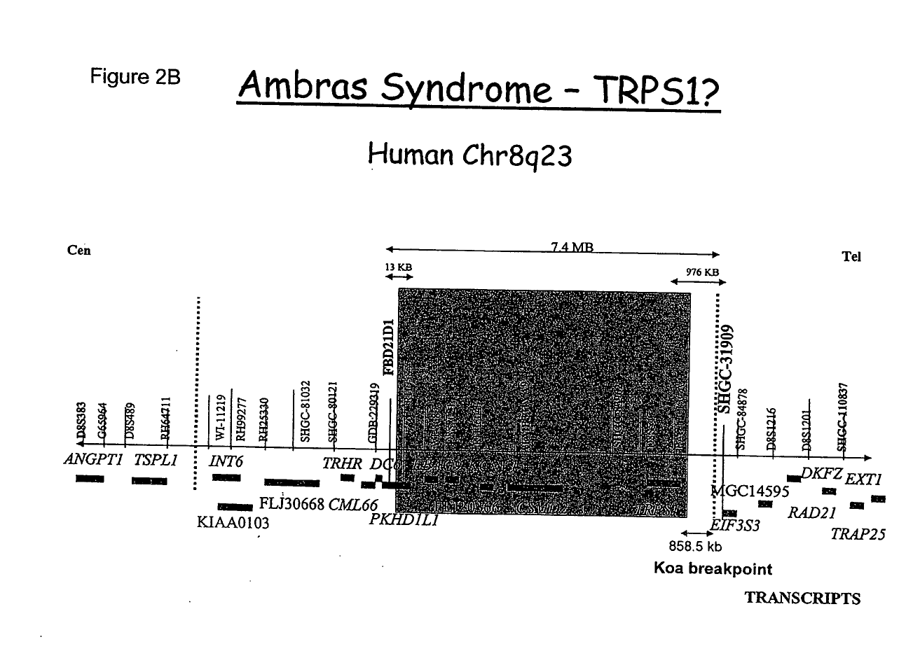

The TRPS1 protein (FIG. 1) is a 1281 amino acid residue protein encoded by a 10,011 bp mRNA with a 3,843 nucleotide long open reading frame. The TRPS1 protein has a predicted isoelectric point of 7.5 and a calculated molecular mass of 141 kDa (Chang et al., 2002, Apoptosis 7:13-21). The mouse and xenopus proteins have 93% and 73% similarity to the human protein. The genomic locus encoding the TRPS1 gene is located on human chromosome 8q23-24 and comprises approximately 260.5 kb of genomic DNA consisting of 7 exons. Several genetic disorders are associated with the 8q23-24 region of the human genome (see FIG. 2A and 2B).

TRPS1 Gene Expression

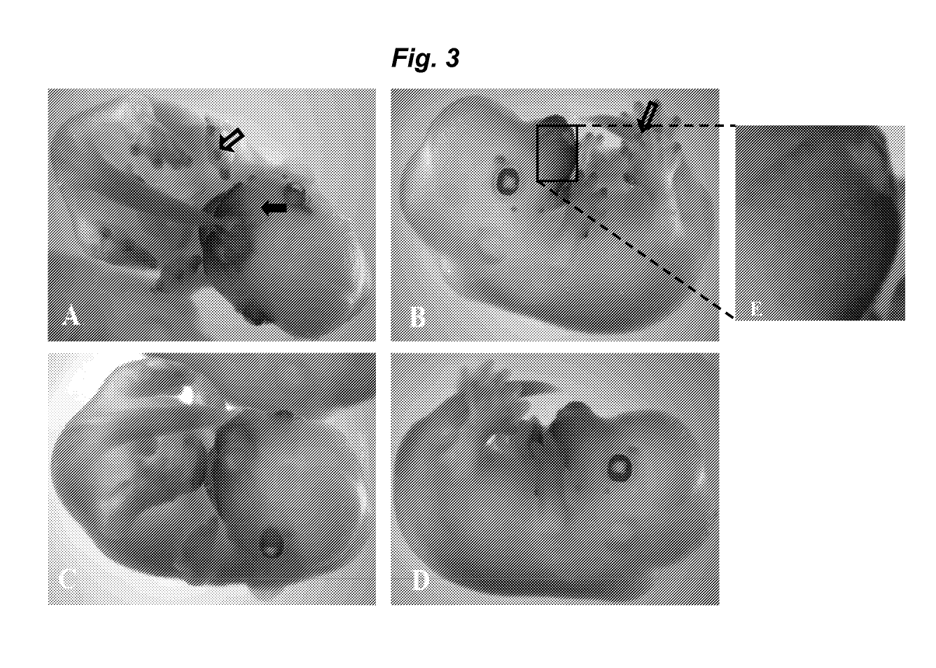

To demonstrate the association between TRPS1 and hair growth, TRPS1 expression was studied in normal mouse embryos using whole mount in situ hybridization utilizing an antisense TRPS1 riboprobe. FIGS. 3A and 3B show a front view and side view, respectively, of a normal mouse embryo probed with antisense TRPS1 probe. Positive signals generated by in situ hybridization of the probe in the regions of the mouse embryo expressing TRPS1 gene sense transcripts shows staining at the phalanges, the mesenchyme surrounding the vibrissae follicles and snout. This data confirms prior data that TRPS1 expression during development reflects the sites of pathology in TRPS1 patients (Malik et al., 2002), and call to mind the anatomical structures affected by tricho-rhino-phalangeal syndrome in humans. FIGS. 3C and 3D show a front and side view, respectively, of a normal mouse embryo probed with control, sense TRPS1 probe. No specific signals are seen, demonstrating that staining with antisense probe (FIG. 3A-B) is specific.

TRPS1 Protein Expression

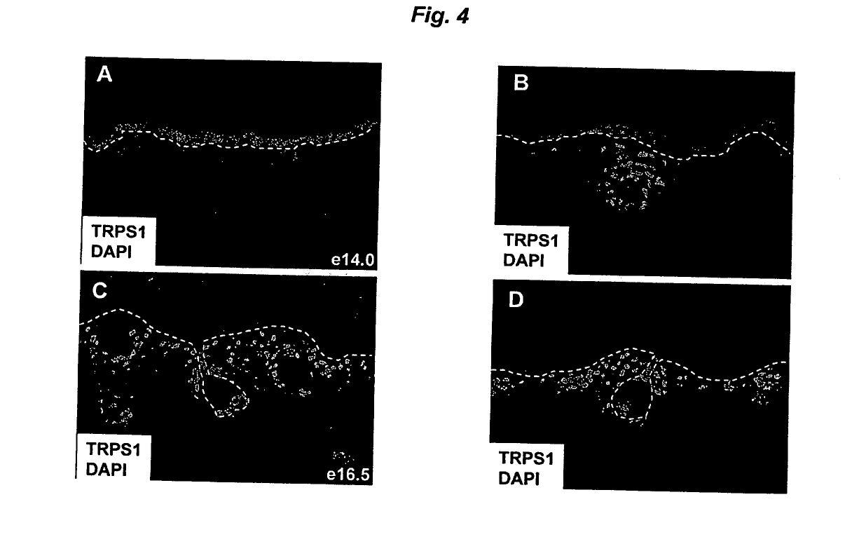

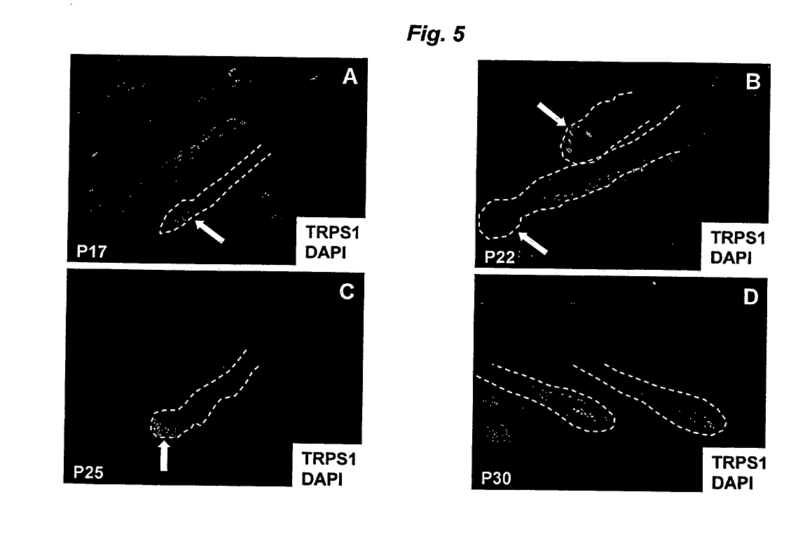

To demonstrate the association of TRPS1 and hair growth at a cellular level, indirect immunofluorescence microscopy was performed using standard techniques (FIG. 4). At e14.0, TRPS1 expression appears as diffuse, spotty staining in the dorsal epidermis (FIG. 4A). During the hair germ stage at e15.5, TRPS1 expression appears in the nuclei of dermal cells in the hair germs, with some diffuse staining still present in the epidermis (FIG. 4B). By the peg stage at e16.5 (FIG. 4C) to e17.5 (FIG. 4D), TRPS1 expression is restricted to the mesenchymal cells surrounding the hair follicle and the cells of the mesenchyme-derived dermal papilla.

Postnatally, TRPS1 continues to be expressed in the dermal papilla, localizing to the nucleus during telogen and anagen (FIG. 5). During catagen at P17, expression is observed as non-nuclear staining in the dermal papilla (FIG. 5A). By early telogen at P22, expression appears nuclear in the dermal papillae of some follicles (FIG. 5B). During late telogen at P25, TRPS1 is expressed in the nuclei of the dermal papilla cells, with additional non-nuclear, punctate staining in the epithelial cells just about the papilla (FIG. 5C). This staining recapitulates the expression observed during morphogenesis, in particular at e15.5, when TRPS1 expression is nuclear in mesenchymal cells and non-nuclear and punctate in the overlying epithelial cells. By postnatal anagen at P30, TRPS1 expression is observed in the nuclei of dermal papilla and hair shaft cortex cells (FIG. 5D). Notably, TRPS1 expression in the mesenchyme surrounding the hair follicle appears to be specific to morphogenesis, as it is not observed during postnatal hair cycling.

In accordance with the immunofluorescence results, TRPS1 expression was shown to decrease to two-fold below e12.5 baseline expression in the epidermis through e14.5 and increase up to four-fold above e13.5 baseline expression in the dermis in a microarray experiment comparing expression patterns in the two compartments.

TRPS1 Expression in TRPS1^{Δgt/Δgt} Knockout Animals

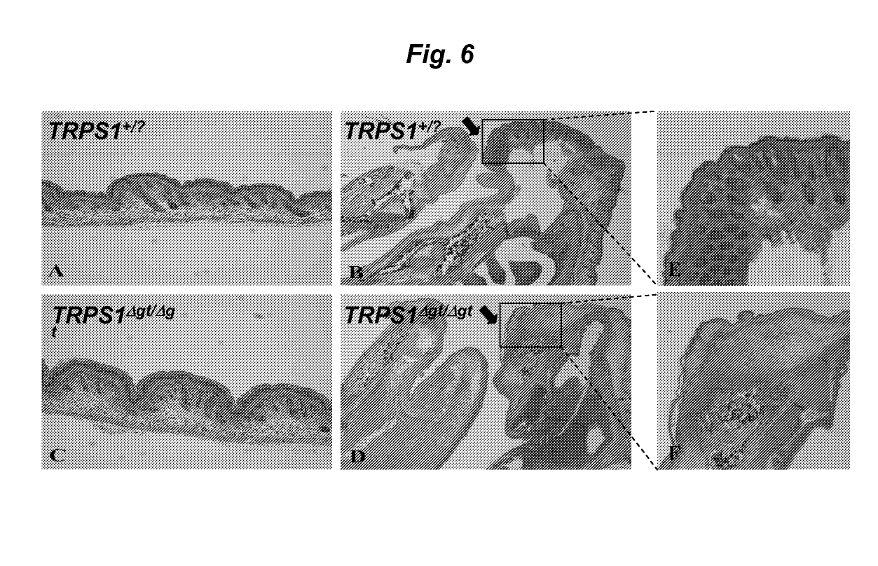

The phenotypic consequences of ablating TRPS1 gene expression in mice was examined by performing comparative histological analyses of mouse relevant tissues from wild-type and TRPS1 knockout (TRPS1^{Δgt/Δgt}) animals. FIGS. 6A and 6B show hematoxylin/eosin (“H&E”) staining stained sections of back-skin and whisker pad, respectively, of a TRPS1 wild-type mouse. Both sections showed normal histology. The stained tissue section from the back-skin of a TRPS1^{Δgt/Δgt} knockout mouse also showed normal histology (FIG. 6C) on analysis after H&E staining. By contrast, a whisker pad section of a TRPS1^{Δgt/Δgt} knockout mouse showed abnormal histology. The whiskers were essentially completely missing as were the hair follicles in the whisker pads (FIG. 6D).

The foregoing data indicates that TRPS1 expression is increased in hair follicles, and particularly the dermal papillae, and is associated with hair growth.

### EXAMPLE 2

### Downstream Targets of TRPS1

To identify downstream targets of TRPS1, microarray hybridization analysis was performed comparing expression patterns in the whisker pads of e12.5 wild-type versus TRPS1^{Δgt/Δgt} mice. The gene list that was generated contained 15 genes that were expressed at higher levels in the wild-type animals, and 18 genes that were expressed at high levels in the TRPS1^{Δgt/Δgt} mice. Because TRPS1 has been shown to specifically bind GATA sites in the DNA (Malik et al., 2001), a search was conducted for GATA-1 sites within 2 kb upstream of the transcriptional start site for the genes identified in the microarray (Table 1).

TABLE 1
Genes identified in microarray comparing expression patterns
in e12.5 wild-type versus TRPS1^{Δgt/Δgt} whisker pads
and the number of GATA-1 binding sites within 2 kb upstream
of their transcriptional start sites.

| | Fold Change | |
|---|---|---|
| Gene | (in WT) | # GATA-1 Sites |
| PR domain containing 1, ZNF domain | 3 | 10 |
| SRY-box containing gene 18 | 3 | 8 |
| Dickkopf homolog 4 | 3 | 5 |
| SRY-box containing gene 21 | 3 | 1 |
| Wnt inhibitory factor 1 | 2 | 2 |
| Lumican | −2 | 6 |
| Tenascin C | −2 | 4 |
| Matrix metalloproteinase 16 | −2 | 6 |
| Decorin | −2 | 8 |
| Calbindin-28K | −4 | 6 |
| Dermatan sulphate proteoglycan 3 | −9 | 12 |

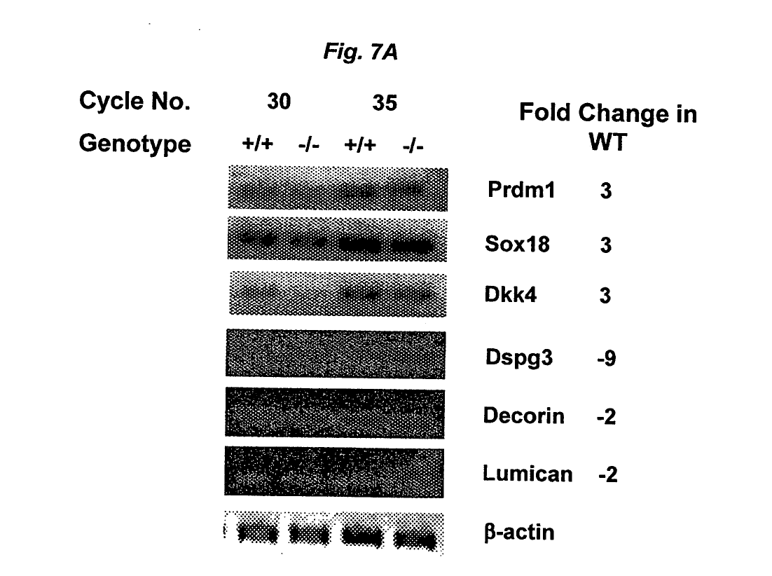

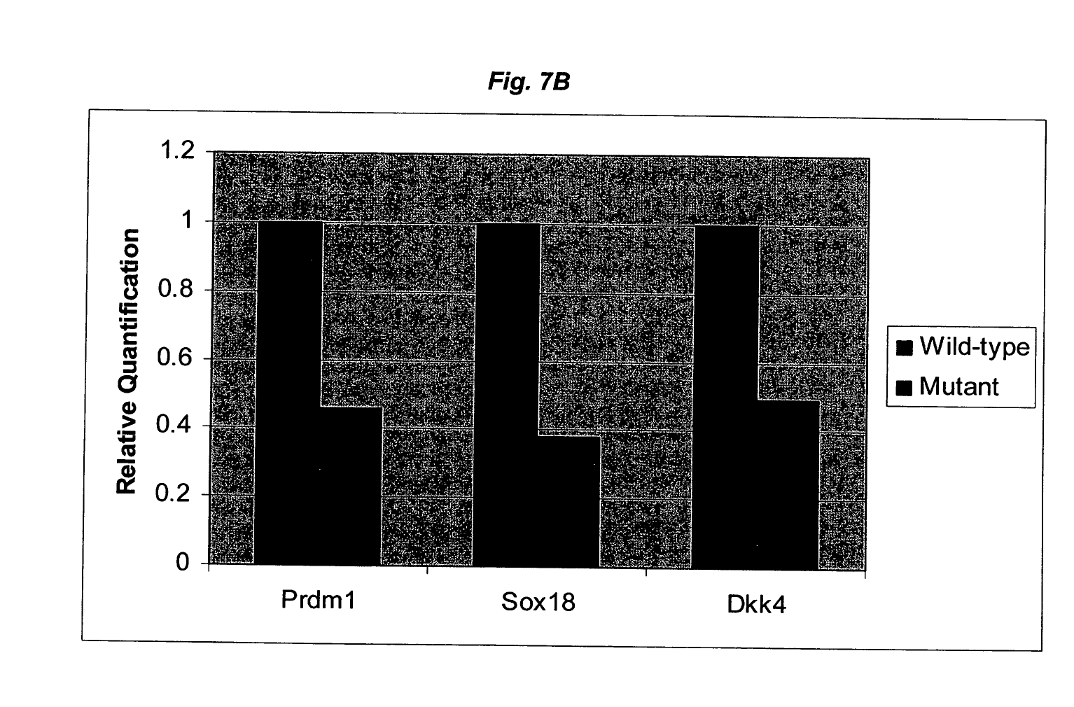

Prdm1, Sox18 and Dkk4 were detected among genes expressed at lower levels in the mutant animals. Sox18 and Prdm1 have been previously localized to the hair follicle dermis and contain 10 and 8 GATA-1 sites, respectively, in their upstream promoters. Dkk4 has recently been localized to the epidermis at sites of epithelial-mesenchymal interactions (Bazzi, unpublished data) and contains 5 GATA-1 sites within 2 kb upstream of the transcriptional start site. The data are consistent with a formulation in which genes that are expressed at higher levels in the wild-type animals are directly or indirectly activated by TRPS1, whereas those expressed at lower levels in the wild-type animals are directly or indirectly repressed by TRPS1. Quantitative RT-PCR analysis confirmed the differential expression of Prdm1, Sox18 and Dkk4 between the wild-type and TRPS1^{Δgt/Δgt} samples, revealing a reduction in expression of at least two-fold in the mutant animals (FIG. 7).

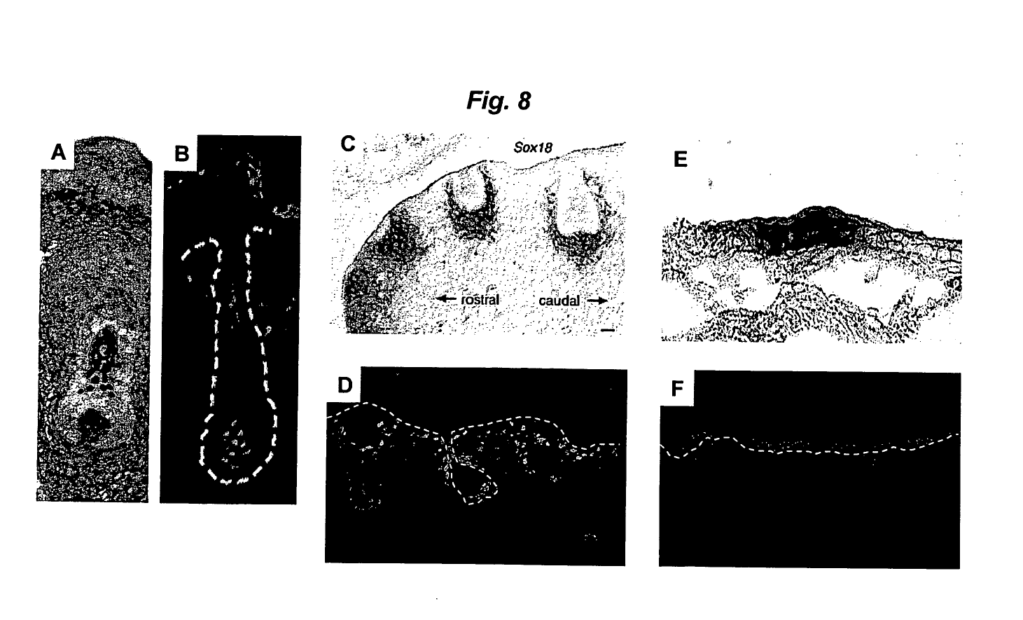

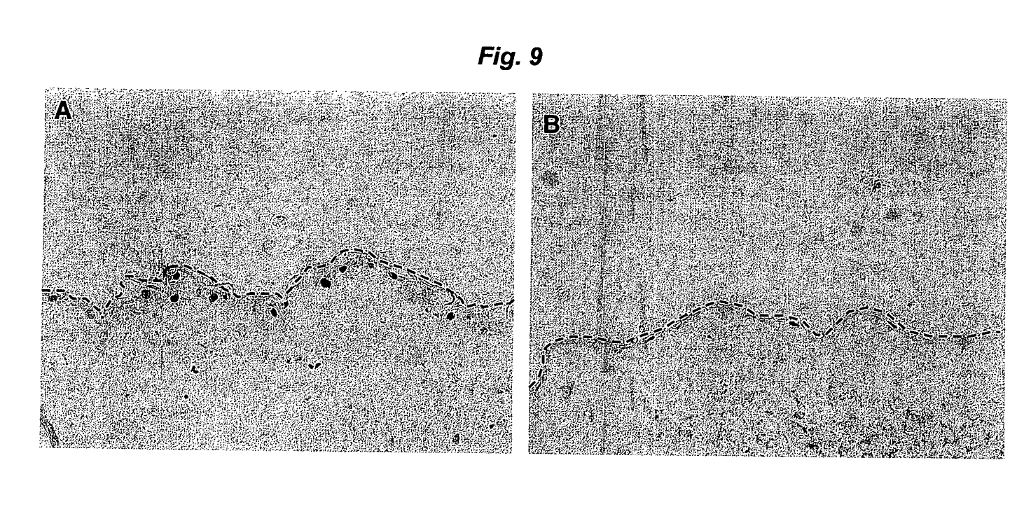

Prdm1 is a Krüppel-type zinc finger protein responsible for terminal differentiation of B lymphocytes (Turner et al., 1994, Cell 77:297-306) and macrophage differentiation (Chang et al., 2000, Nat. Immunol. 1: 169-176). Targeted disruption of the gene results in premature death at e10.5 (Vincent et al., 2005, Development 132:1315-1325). Prdm1 is expressed in the granular layer of the skin and the inner root sheath (IRS) precursors and dermal papilla of the hair follicle (Chang and Calame, 2002, Mech. Dev., 117:305-309). Prdm1 and TRPS1 have overlapping expression patterns in the dermal papilla (FIG. 8A, 8B). Immunohistochemistry performed on P1 whisker pads of wild-type and TRPS1^{Δgt/Δgt} mice revealed a significant decrease in the expression of Prdm1 in the granular layer of the epidermis in mutant animals (FIG. 9).

Sox18 is an SRY-related transcription factor (Hosking et al., 1995, Nucleic Acids Res., 23:2626-2628) expressed in the underlying mesenchyme of hair follicles during mouse embryogenesis (Pennisi et al. 2000, Nat. Genet. 24:434-437). Mutations in Sox18 are responsible for the ragged (Ra) mouse phenotype, characterized by varying degrees of coat sparseness. Heterozygotes have short vibrissae and a thin, ragged coat, whereas homozygotes almost completely lack vibrissae and coat hairs (Carter and Phillips, 1954, J. Hered, 45:151-15). In humans, mutations in SOX18 result in hypotrichosis-lymphedema-telangiectasia syndrome, characterized by sparse scalp hair and the absence of eyebrows and eyelashes (Irrthum et al., 2003, Am. J. Hum. Genet., 72:1470-1478). During murine morphogenesis, Sox18 and TRPS1 have overlapping expression in the mesenchyme surrounding the hair follicle (FIG. 8C, 8D).

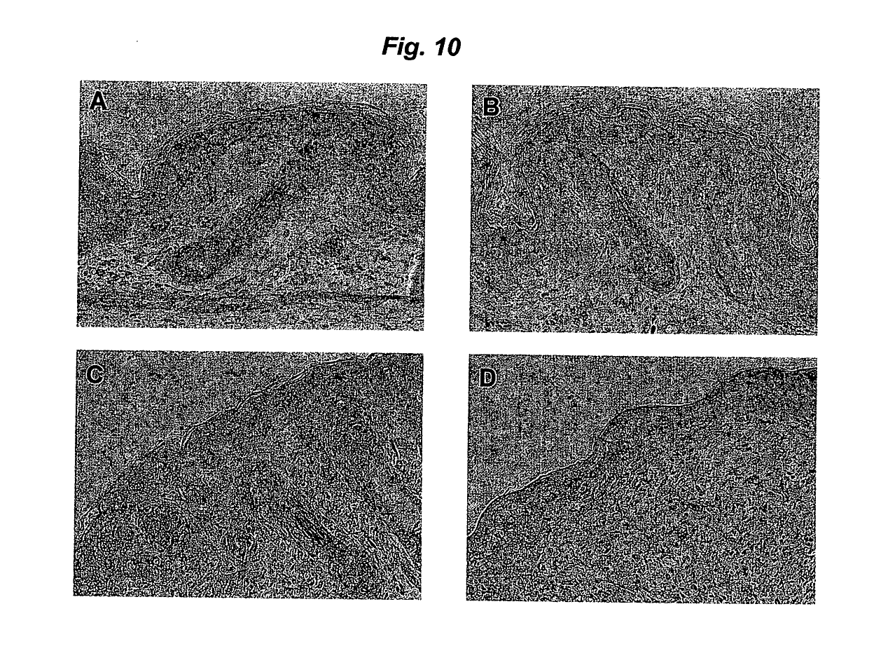

Dkk4 is a secreted Wnt antagonist that acts similar to Dkk1 in its ability to inhibit Wnt signaling (Mao and Niehrs, 2003, Gene 302:179-183). Dkk4 has recently been localized to the epidermis at sites of epithelial-mesenchymal interactions during mouse embryogenesis (Bazzi, unpublished data). At the onset of hair follicle morphogenesis, Dkk4 and TRPS1 have overlapping expression in the epidermis (FIG. 8E, 8F). Immunohistochemistry performed on P1 dorsal skin of wild-type and TRPS1^{Δgt/Δgt} mice revealed a decrease in the expression of Dkk4 in the hair shaft cuticle of mutant animals (FIG. 10).

### EXAMPLE 3

### TRPS1 Overexpression

The data are consistent with a formulation in which Prdm1, Sox18 and Dkk4 are regulated by TRPS1 in the developing hair follicle. To determine the effect of overexpression of TRPS1 on the promoters of Prdm1, Sox18 and Dkk4, promoter assays are performed in both primary human fibroblast and HaCat human keratinocyte cells lines using a luciferase reporter. Electrophoretic mobility shift assays are used to find direct targets of TRPS1. For those functional TRPS1 targets identified, siRNA is used to knock them down in primary mouse dermal cultures to place TRPS1 and its interaction partners within specific signaling pathways.

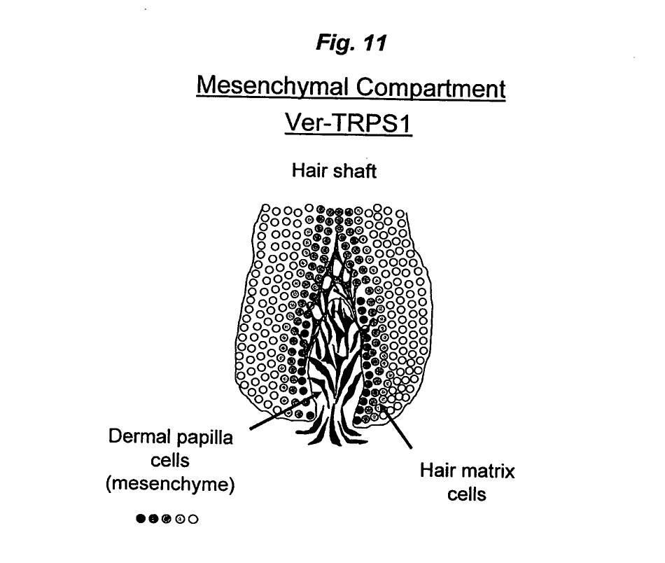

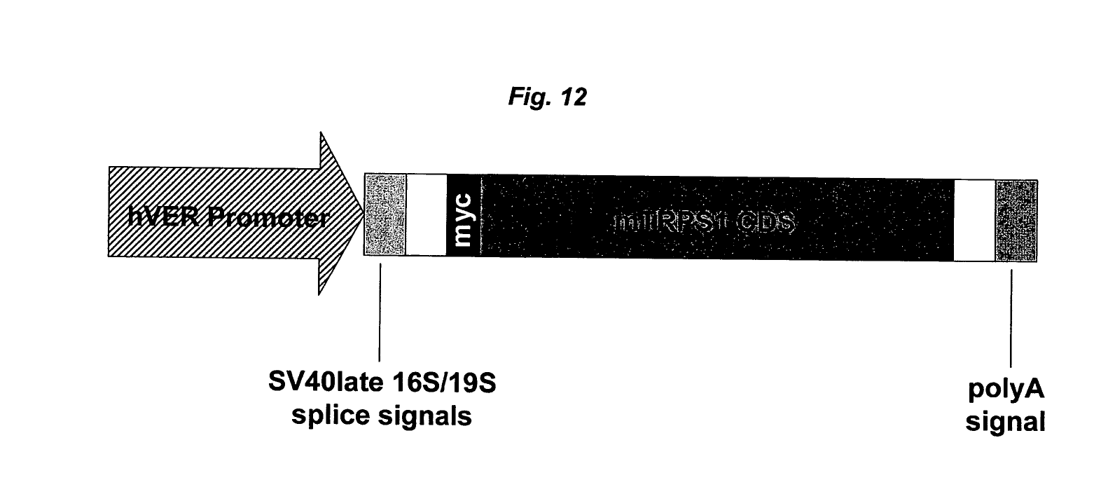

To evaluate the role of TRPS1 overexpression in hypertrichosis and to recapitulate Ambras syndrome, transgenic mice are generated that overexpress TRPS1 under the hair follicle mesenchyme-specific versican promoter (Kishimoto et al., 1999, Proc. Natl. Acad. Sci. USA., 96:7336-7341) to characterize the effects of TRPS1 overexpression on hair follicle morphogenesis and cycling (FIG. 11). The versican promoter (FIG. 12) has been demonstrated to drive expression of a lacZ reporter gene in the mesenchymal condensate of transgenic mice during embryogenesis and the dermal papilla of adult transgenic mice during anagen (Kishimoto et al., 1999, supra).

Detailed phenotypic analyses are performed to determine the effects of TRPS1 overexpression in the mesenchymal compartment at different time points in the hair cycle. First, standard histochemical methods are employed on longitudinally sectioned hair follicles to identify stage-specific follicle defects in transgenic compared to age-matched wild-type mice. Alkaline phosphatase staining is used to identify the dermal papilla throughout the hair cycle. Oil-red-O staining is used to examine the sebaceous gland and hair canal during mid-anagen. Finally, TUNEL staining is used to study the hair matrix, outer root sheath and inner root sheath during early catagen (Muller-Rover et al., 2001, J. Invest. Dermatol., 117:3-15). Any abnormalities in the morphology or location of these cell populations during the hair cycle due to TRPS1 overexpression are identified with these assays.

Expression of specific molecules involved in hair follicle morphogenesis and cycling are examined, including TGFβRII, NCAM, P-cadherin, nexin-1, β-catenin, LEF1, SHH, and Ltbp1 (Muller-Rover et al., 2001, supra; Stenn and Paus, 2001, Physiological Rev., 81:449-494; Millar, 2002, J. Invest. Dermatol., 118:216-225). The expression of these molecules is compared between transgenic and wild-type skin samples by quantitative RT-PCR analysis, in situ hybridization, Western blot analysis, and immunohistochemistry. Electron microscopy and whole mount hair follicle in situ hybridization is used to characterize the effects of TRPS1 overexpression in the mesenchymal compartment.

Various publications are cited herein, the contents of which are hereby incorporated by reference in their entireties.

I claim:

1. A method of promoting hair growth in a subject comprising increasing the level and/or activity of TRPS1 mRNA and/or protein in a cell of the subject.

2. The method of claim 1, wherein the cell is a hair follicle cell.

3. The method of claim 2, wherein the hair follicle cell is a dermal papilla cell.

4. The method of claim 1, wherein the level of TRPS1 is increased by introducing, into a cell of a subject, TRPS1 mRNA or protein.

5. The method of claim 1, wherein the level and/or activity of TRPS1 is increased by providing an agent that results in increased expression of an endogenous TRPS1 gene, increased functional activity of a TRPS1 protein, or increased expression and/or activity of a target of a TRPS1 protein.

6. The method of claim 1, wherein the target of a TRPS1 protein is selected from the group consisting of Prdm1, Sox18 and Dkk4.

7. A method of inhibiting hair growth in a subject comprising decreasing the level and/or activity of TRPS1 mRNA and/or protein in a cell of the subject.

8. The method of claim 7, wherein the cell is a hair follicle cell.

9. The method of claim 8, wherein the hair follicle cell is a dermal papilla cell.

10. The method of claim 7, wherein the level and/or activity of TRPS1 is decreased by providing an agent that results in decreased expression of an endogenous TRPS1 gene, decreased functional activity of a TRPS1 protein, or decreased expression and/or activity of a target of a TRPS1 protein.

11. The method of claim 10, wherein the agent is selected from the group consisting of a catalytic nucleic acid, a siRNA and an antisense oligonucleotide directed to the endogenous TRPS1 gene.

12. The method of claim 10, wherein the agent is selected from the group consisting of a catalytic nucleic acid, a siRNA and an antisense oligonucleotide directed to a target of a TRPS1 protein.

13. The method of claim 12, wherein the target of TRPS1 is selected from the group consisting of Prdm1, Sox18 and Dkk4.

14. The method of claims 1 or 7, wherein the subject is a human subject.

15. A method for identifying agents used to inhibit hair growth, comprising
a. exposing an appropriate test cell or organism to a test agent, and
b. determining whether expression of TRPS1 is decreased relative to the level of TRPS1 in a control cell or organism not exposed to the test agent.

16. A method for identifying agents used to promote hair growth, comprising
a. exposing an appropriate test cell or organism to a test agent, and
b. determining whether expression of TRPS1 is increased relative to the level of TRPS1 in a control cell or organism not exposed to the test agent.

17. A transgenic non-human animal containing a transgene comprising a TRPS1 gene, operably linked to a promoter element, wherein the promoter element may be constitutively active or inducible in hair follicle cells of the animal.

18. The transgenic animal of claim 17, wherein the transgene encodes an antisense or sense TRPS1 gene.

19. The transgenic animal of claim 17, wherein the transgene comprises a TRPS1 gene, and wherein the promoter element is a versican promoter.

20. The transgenic animal of claim 17, wherein the transgene interrupts or disrupts expression of at least one TRPS1 gene.

21. The transgenic animal of claim 20, wherein the transgenic animal is a TRPS1 knockout animal.

22. The transgenic animal of claim 21, wherein the TRPS1 knockout animal contains a deletion of the nucleic acid sequence encoding a GATA binding region of TRPS1.

23. An isolated cell of the transgenic animal of claim 17.

24. A composition used to inhibit hair growth, comprising an agent that inhibits expression of TRPS1 or the expression of a target of TRPS1.

25. The composition of claim 24, wherein the agent is selected from the group consisting of an antisense oligonucleotide, a siRNA, and a catalytic nucleic acid directed to TRPS1.

26. The composition of claim 24, wherein the agent is selected from the group consisting of an antisense oligonucleotide, a siRNA, or a catalytic nucleic acid directed to a target of TRPS1.

27. The composition of claim 24, wherein the target of TRPS1 is Prdm1, Sox18 or Dkk4.

28. A composition used to promote hair growth, comprising an agent that increases TRPS1 expression or the expression of a target of TRPS1.

29. The composition of claim 28, wherein the agent comprises a TRPS1 nucleic acid and/or protein, an agent that increases TRPS1 expression, and/or hair follicle cells in which TRPS1 expression is increased or which have been administered TRPS1 protein.

30. The composition of claim 28, wherein the target of TRPS1 is selected from the group consisting of Prdm1, Sox18 and Dkk4.

31. An assay system for identify agents that increase or decrease TRPS1 expression, comprising the transgenic animal of claim 17 or the isolated cell of claim 23.

32. A method of promoting hair growth in a subject comprising increasing the level and/or activity of a target of TRPS1.

33. The method of claim 32, wherein the target of TRPS1 is selected from the group consisting of Prdm1, Sox18 and Dkk4.

34. A method of inhibiting hair growth in a subject comprising decreasing the level and/or activity of a target of TRPS1.

35. The method of claim 34, wherein the level and/or activity is decreased by providing an agent that results in decreased level of a target of TRPS1.

36. The method of claim 34, wherein the target of TRPS1 is selected from the group consisting of Prdm1, Sox18 and Dkk4.

37. The method of claim 35, wherein the agent is a selected from the group consisting of a catalytic nucleic acid, an antisense oligonucleotide and a siRNA directed to a target of TRPS1.

38. A method for increasing TRPS1 expression in a cell by introducing, into a cell, a nucleic acid selected from the group consisting of (i) SEQ ID NO:3; (ii) a sequence 90 percent homologous to SEQ ID NO:3; and (iii) a sequence which encodes a TRPS1 protein with amino acid sequence SEQ ID NO:2.

39. The method of claim 38, wherein the nucleic acid is operably linked to a promoter element.

40. The method of claim 39, wherein the promoter element is selectively active in a hair follicle cell.

41. The method of claim 39, wherein the promoter element is inducible.

42. The method of claim 38, wherein the nucleic acid is SEQ ID NO:3.

43. A composition used to increase TRPS1 expression in a cell, comprising a nucleic acid selected from the group consisting of (i) SEQ ID NO:3; (ii) a sequence 90 percent homologous to SEQ ID NO:3; and (iii) a sequence which encodes a TRPS1 protein with amino acid sequence SEQ ID NO:2.

44. The composition of claim 43, wherein the nucleic acid is operably linked to a promoter element.

45. The composition of claim 43, wherein the composition is a topical formulation or a pharmaceutical composition.

46. The composition of claim 43, wherein the nucleic acid is SEQ IS NO:3.

47. A method for increasing TRPS1 mRNA and/or protein in a cell, comprising administering an agent that results in an increase in that results in increased expression of an endogenous TRPS1 gene or increased functional activity of a TRPS1 protein.

48. The method of claim 47, wherein the agent is selected from the group consisting of a small molecule, a peptide, a protein, a nucleic acid, a lipid, or a carbohydrate.

49. The method of claim 47, wherein the agent is selected from the group consisting of an androgen antagonist and a RNF4 antagonist.
